SEQUENCE LISTING (47 sequences)

SEQ ID NO: 1 - protein, 58 aa, synthetic construct
AEAKYAKEQQNAFYEILHLPNLTEEQRNAFIQKLKDDPSQSSELLSEAKKLNDSQAPK

SEQ ID NO: 2 - protein, 58 aa, synthetic construct
  SITE 6: note = Y, N, S, T, D, or A
  SITE 9: note = L, R, K, W, D, I, N, or Q
  SITE 10: note = H, A, S, F, I, D, E, Q, T, N, or L
  SITE 11: note = T, Y, D, A, L, P, I, or N
  SITE 13: note = I, Y, L, V, T, or F
  SITE 14: note = Q, Y, L, V, G, F, E, A, M, or S
  SITE 15: note = V, S, Q, or E
  SITE 17: note = H, A, V, G, R, I, F, or L
  SITE 18: note = S, Y, L, G, T, N, M, D, R, W, E, or H
  SITE 23: note = T or A
  SITE 24: note = L, K, A, G, R, H, W, V, or E
  SITE 25: note = D, P, T, N, V, S, H, L, R, A, W, or E
  SITE 27: note = R, K, T, I, or A
  SITE 28: note = A, Y, R, N, D, Q, I, H, L, S, or F
  SITE 31: note = L, K, I, or T
  SITE 32: note = L, G, Y, R, A, F, V, S, or Q
  SITE 35: note = A or K
  SITE 35: note = F, S, V, H, Y, D, E, or K
  SITE 36: note = N or D
  SITE 44: note = L or F
  SITE 44: note = S or P
  SITE 49: note = R or K
AEAKYXKEXXXAXXXIXXLPNLXXXQXXAFXXXLXXDPSQSSELXXEAXKLNDSQAPK

SEQ ID NO: 3 - protein, 58 aa, synthetic construct
AEAKYYKELHNAIGVIRSLPNLTRDQRAAFLLALFNDPSQSSELLSEAKKLNDSQAPK

SEQ ID NO: 4 - protein, 58 aa, synthetic construct
AEAKYYKELHNAIQVIHYLPNLTLDQKYAFKLALFNDPSQSSELLSEAKKLNDSQAPK

SEQ ID NO: 5 - protein, 58 aa, synthetic construct
AEAKYNKELATAYYVIALLPNLTEPQRAAFLGALSDDPSQSSELLSEAKKLNDSQAPK

SEQ ID NO: 6 - protein, 58 aa, synthetic construct
AEAKYYKELHTAILSIHGLPNLTKTQKRAFIYALFNDPSQSSELLSEAKKLNDSQAPK

SEQ ID NO: 7 - protein, 58 aa, synthetic construct
AEAKYNKERSYAIYQIVLLPNLTANQRNAFLRALVDDPSQSSELLSEAKKLNDSQAPK

SEQ ID NO: 8 - protein, 58 aa, synthetic construct
AEAKYSKEKFDAILSILSLPNLTAVQRDAFLGALHDDPSQSSELLSEAKKLNDSQAPK

SEQ ID NO: 9 - protein, 58 aa, synthetic construct
AEAKYYKELHYAIVSIRGLPNLTASQRIAFLFALFNDPSQSSELLSEAKKLNDSQAPK

SEQ ID NO: 10 - protein, 58 aa, synthetic construct
AEAKYTKEWIAAYLQIGYLPNLTGVQRQAFTAALYNDPSQSSELLSEAKKLNDSQAPK

SEQ ID NO: 11 - protein, 58 aa, synthetic construct
AEAKYDKEKSNAILSIVTLPNLTRVQRHAFLVALHNDPSQSSELLSEAKKLNDSQAPK

SEQ ID NO: 12 - protein, 58 aa, synthetic construct
AEAKYYKELHDAIVQIRNLPNLTRPQRAAFKLALFNDPSQSSELLSEAKKLNDSQAPK

SEQ ID NO: 13 - protein, 58 aa, synthetic construct
AEAKYYKELHNAIGVIRSLPNLTRHQRAAFLLALFNDPSQSSELLSEAKKLNDSQAPK

SEQ ID NO: 14 - protein, 58 aa, synthetic construct
AEAKYDKERLNAIFQIVLLPNLTRDQKLAFTAALYDDPSQSSELLSEAKKLNDSQAPK

SEQ ID NO: 15 - protein, 58 aa, synthetic construct
AEAKYYKELHNAIQVIHYLPNLTRDQKYAFKLALFNDPSQSSELLSEAKKLNDSQAPK

SEQ ID NO: 16 - protein, 58 aa, synthetic construct
AEAKYYKELHNAIGVIRSLPNLTRDQRYAFKLALFNDPSQSSELLSEAKKLNDSQAPK

SEQ ID NO: 17 - protein, 58 aa, synthetic construct
AEAKYYKELHNAIGVIRSLPNLTHHQRSAFLLALFNDPSQSSELLSEARKLNDSQAPK

SEQ ID NO: 18 - protein, 58 aa, synthetic construct
AEAKYYKELHNAIGVIRSLPNLTHHQKSAFLLALFNDPSQSSELLPEAKKLNDSQAPK

SEQ ID NO: 19 - protein, 58 aa, synthetic construct; identical to SEQ ID NO: 13

SEQ ID NO: 20 - protein, 58 aa, synthetic construct
AEAKYYKELHDAIQVIHYLPNLTLDQKYAFKLALFNDPSQSSELLSEAKKLNDSQAPK

SEQ ID NO: 21 - skipped

SEQ ID NO: 22 - protein, 58 aa, synthetic construct
AEAKYAKELDNAYYEIVTLPNLTWLQTFAFIFALDNDPSQSSELLSEAKKLNDSQAPK

SEQ ID NO: 23 - protein, 58 aa, synthetic construct
AEAKYAKELHNAIGVIRSLPNLTRHQRAAFLLALFNDPSQSSELLSEAKKLNDSQAPK

SEQ ID NO: 24 - protein, 58 aa, synthetic construct
AEAKYYKELHDAIQVIHYLPNLTRRQRAAFKLALFNDPSQSSELLSEAKKLNDSQAPK

SEQ ID NO: 25 - protein, 58 aa, synthetic construct
AEAKYAKEKSNAVLSIVSLPNLTRAQRHAFLVALHNDPSQSSELLSEAKKLNDSQAPK

SEQ ID NO: 26 - protein, 58 aa, synthetic construct
AEAKYNKEDSNAYEEIIMLPNLTVWQILAFIVALDNDPSQSSELLSEAKKLNDSQAPK

SEQ ID NO: 27 - protein, 58 aa, synthetic construct
AEAKYDKELELAYLEIFDLPNLTWLQTFAFISALENDPSQSSELLSEAKKLNDSQAPK

SEQ ID NO: 28 - protein, 58 aa, synthetic construct
AEAKYAKELEPALAEILRLPNLTWLQTIAFILALEDDPSQSSELLSEAKKLNDSQAPK

SEQ ID NO: 29 - protein, 58 aa, synthetic construct
AEAKYYKEKSNAVLSIVSLPNLARAQRAAFLVALHDDPSQSSEFLSEAKKLNDSQAPK

SEQ ID NO: 30 - protein, 58 aa, synthetic construct
AEAKYAKEDQNALSEILWLPNLTGWQTFAFILALENDPSQSSELLSEAKKLNDSQAPK

SEQ ID NO: 31 - protein, 58 aa, synthetic construct
AEAKYYKELHNAIGVIRSLPNLTRHQRTAFLLALFNDPSQSSELLSEAKKLNDSQAPK

SEQ ID NO: 32 - protein, 58 aa, synthetic construct
AEAKYAKEIEIAYVEILLLPNLTWLQTYAFIFALEDDPSQSSELLSEAKKLNDSQAPK

SEQ ID NO: 33 - protein, 58 aa, synthetic construct
AEAKYAKENTIALMEIIELPNLTLLQTLAFIVALDDDPSQSSELLSEAKKLNDSQAPK

SEQ ID NO: 34 - protein, 58 aa, synthetic construct
AEAKYDKELNIATMEILTLPNLTLWQAIAFIVALDDDPSQSSELLSEAKKLNDSQAPK

SEQ ID NO: 35 - protein, 15 aa, synthetic construct
GSGGGSGGGKGGGGT

SEQ ID NO: 36 - protein, 15 aa, synthetic construct
GGGGSGGGGSGGGGS

SEQ ID NO: 37 - protein, 18 aa, synthetic construct
GSTSGSGKPGSGEGSTKG

SEQ ID NO: 38 - protein, 20 aa, synthetic construct
PSGQAGAAASESLFVSNHAY

SEQ ID NO: 39 - protein, 7 aa, synthetic construct
EASGGPE

SEQ ID NO: 40 - protein, 38 aa, synthetic construct
AEAAKEAAKEAAKEAAKALEAEAAKEAAKEAAKEAAKA

SEQ ID NO: 41 - protein, 10 aa, synthetic construct
AEAAKEAAKA

SEQ ID NO: 42 - protein, 22 aa, synthetic construct
SGGGGSGGGGSGGGGSGGGGSG

SEQ ID NO: 43 - protein, 10 aa, synthetic construct
GGGGSGGGGS

SEQ ID NO: 44 - protein, 18 aa, synthetic construct
GSTSGSGKPGSGEGSTKG

SEQ ID NO: 45 - protein, 15 aa, synthetic construct
EPKSSDKTHTSPPSP

SEQ ID NO: 46 - protein, 25 aa, synthetic construct
RATPSHNSHQVPSAGGPTANSGTSG

SEQ ID NO: 47 - protein, 19 aa, synthetic construct
SSGGGGSGGGGGGSSRSSL

(19) United States
(12) Patent Application Publication
(10) Pub. No.: US 2025/0101109 A1
(43) Pub. Date: Mar. 27, 2025

(54) ANTI-B7-H3 COMPOUNDS AND METHODS OF USE

(71) Applicant: REGENTS OF THE UNIVERSITY OF MINNESOTA, Minneapolis, MN (US)

(72) Inventors: Benjamin Hackel, Edina, MN (US); Abbigael Harthorn, Minneapolis, MN (US)

(21) Appl. No.: 18/832,026

(22) PCT Filed: Jan. 24, 2023
(86) PCT No.: PCT/US2023/011458

§ 371 Date: Jul. 22, 2024

Related U.S. Application Data
(60) Provisional application No. 63/302,290, filed on Jan. 24, 2022.

(51) Int. Cl. C07K 16/28; A61K 47/68; A61K 51/10

(52) U.S. Cl. CPC C07K 16/2827; A61K 47/6849; A61K 51/1027; A61K 2123/00; C07K 2317/92; C07K 2317/94

(57) ABSTRACT

Anti-B7-H3 compounds include an anti-B7-H3 affibody. The anti-B7-H3 compounds may include one or more functional components such as a targeting component, an imaging component, an enzyme, or a small molecule drug. Compositions including an anti-B7-H3 may be administered to a subject. The subject may have or be at risk of having a tumor or cancer.

### CROSS-REFERENCE TO RELATED APPLICATIONS

This application claims the benefit of U.S. Provisional Application Ser. No. 63/302,290, filed on Jan. 24, 2022, which is incorporated by reference herein in its entirety.

### GOVERNMENT FUNDING

This invention was made with government support under EB023339 awarded by the National Institutes of Health. The government has certain rights in the invention.

### SEQUENCE LISTING

This application contains a Sequence Listing electronically submitted to the United States Patent and Trademark Office via Patent Center as an XML file entitled “0110_000692WO01.xml” having a size of 49.6 kilobytes and created on Jan. 22, 2023. Due to the electronic filing of the Sequence Listing, the electronically submitted Sequence Listing serves as both the paper copy required by 37 CFR § 1.821 (c) and the CRF required by § 1.821 (e). The information contained in the Sequence Listing is incorporated by reference herein.

### SUMMARY

This disclosure describes, in one aspect, an anti-B7-H3 compound. The anti-B7-H3 compound includes an anti-B7-H3 affibody. The anti-B7-H3 affibody includes the amino acid sequence of SEQ ID NO:2, an amino acid sequence having at least 90% sequence similarity to SEQ ID NO:2, or an amino acid sequence having at least 90% sequence identity to SEQ ID NO: 2.

In one or more embodiments, the anti-B7-H3 affibody includes any one of the amino acid sequences of SEQ ID NO:3; SEQ ID NO:4, SEQ ID NO:5, SEQ ID NO:6, SEQ ID NO:7, SEQ ID NO: 8, SEQ ID NO:9, SEQ ID NO: 10; SEQ ID NO:11, SEQ ID NO: 12, SEQ ID NO: 13; SEQ ID NO: 14, SEQ ID NO:15; SEQ ID NO: 16, SEQ ID NO: 17, SEQ ID NO:18, SEQ ID NO: 19, SEQ ID NO:20, SEQ ID NO:22, SEQ ID NO:23, SEQ ID NO:24, SEQ ID NO:25, SEQ ID NO: 26, SEQ ID NO:27, SEQ ID NO:28, SEQ ID NO:29, SEQ ID NO:30, SEQ ID NO: 31, SEQ ID NO: 32, SEQ ID NO:33, SEQ ID NO:34, an amino acid sequence having at least 90% sequence similarity to any of the listed sequences, or an amino acid sequence having at least 90% sequence identity to any of the listed sequences.

In one or more embodiments, the anti-B7-H3 compound includes a first functional component operably coupled to the B7-H3 affibody. In one or more embodiments, the first functional component includes a targeting component, an imaging component, an enzyme, or a small molecule drug.

In one or more embodiments, the anti-B7-H3 compound may include a second functional component operably couple to the anti-B7-H3 affibody. In one or more embodiments, the second functional component includes a targeting component, an imaging component, an enzyme, or a small molecule drug. In one or more embodiments, the first functional component is directly linked to the anti-B7-H3 affibody and the second functional component is directly linked to the anti-B7-H3 affibody. In one or more embodiments, the first functional component is directly linked to the anti-B7-H3 affibody and the second functional component is directly linked to the first functional component.

In another aspect, the present disclosure describes a composition including an anti-B7-H3 compound and a pharmaceutically acceptable carrier.

In another aspect, the present disclosure describes a method that includes administering a composition that includes an anti-B7-H3 compound to a subject. In one or more embodiments, the subject has a tumor. In one or more embodiments, the subject has cancer. In one or more embodiments, the composition is administered prior to, simultaneously with, or following chemotherapy, surgical resection of a tumor, or radiation therapy. In one or more embodiments, the method further includes detecting the imaging component to detect the anti-B7-H3 compound bound to B7-H3 expressed by a cell. In one or more embodiments, the cell is a cancer cell.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Enriched affibody (ABY) binders to B7-H3. (A) Yeast underwent three magnetic-activated cell sorting (MACS) selections (populations 0.1, 0.2, 0.3 on x-axis) with soluble B7-H3 extracellular domain, showing enrichment to B7-H3 target over negative Renilla reniformis green fluorescent protein (rrGFP) and avidin beads. The enriched ABY population underwent a fluorescent-activated cell sorting (FACS) selection to B7-H3 (yielding population 0.4 on x-axis). ND indicates no data, as binding to avidin beads and negative rrGFP were not analyzed in this sort. The 0.4 population ABY library was extracted and underwent error-prone PCR and then was electroporated back into yeast. The mutated naïve library underwent a MACS selection (yielding population 1.1. on x-axis), followed by a monovalent MACS selection (yielding population 1.2 on x-axis) with 100 nM recombinant human B7-H3. Remaining selections were done via FACS (to achieve populations 1.3 and 1.4). (B) Following three MACS selections, yeast were incubated with 50 nM recombinant human B7-H3 extracellular domain followed by streptavidin-Alexa Fluor 647. ABY display was quantified with mouse an anti-c-Myc antibody and goat anti-mouse-Alexa Fluor 488. Flow cytometric analysis of 10,000 random variants is shown (population 0.4). (C) The enriched population was labeled with biotin-B7-H3 cell lysate, streptavidin-AF647, and chicken anti-c-Myc-FITC. Collected cells yielded population 1.4. The boxes in (B) and (C) are the cells collected during the FACS sort.

FIG. 2. Protease and thermostability of enriched ABY population to yield population 1.5. (A) The affibody library was incubated with 1×10⁻⁶ Units/μL proteinase K for 10 minutes at 55° C., followed by incubation with biotinylated detergent solubilized MS1-B7-H3 cell lysate and labeled with streptavidin-Alexa Fluor 647. The top 0.5% of B7-H3 binders were selected via flow cytometry (gray box). (B) The affibody library was incubated with (+Protease) or without (Control) proteinase K at 55° C. before incubation with biotinylated B7-H3 lysate. The small decrease in B7-H3 binding of the +protease sample, as compared to the control, indicates some variants were susceptible to proteinase K and increased temperature.

FIG. 3. Sequences of evolved B7-H3-binding affibodies in the 0.4, 1.4, and 1.5 populations. ABY025 indicates the affibody sequence (SEQ ID NO:18) that the affibodies of the present disclosure were derived from (see Design of an Optimized Scaffold for Affibody Molecules, 2010 (doi: 10.1016/j.jmb.2010.03.002). The affibody library design is also shown. Eight colonies were sequenced in the 0.4 population (0.4.1=SEQ ID NO:4; 0.4.2=SEQ ID NO: 5; 0.4.3=SEQ ID NO:6; 0.4.4=SEQ ID NO:7; 0.4.5=SEQ ID NO:8; 0.4.6=SEQ ID NO: 9; 0.4.7=SEQ ID NO:10), where 0.4.2 was observed twice (indicated by “×2”). Population shows diverse amino acid sequence among variants. Ten colonies were sequenced in the 1.4 population (1.4.1=SEQ ID NO:11; 1.4.2=SEQ ID NO: 12; 1.4.3=SEQ ID NO:13; 1.4.4=SEQ ID NO: 14; 1.4.5=SEQ ID NO: 15; 1.4.6=SEQ ID NO: 16; 1.4.7=SEQ ID NO: 17; 1.4.8=SEQ ID NO:18), where 1.4.1 and 1.4.2 were observed twice (indicated by “×2”). Seven colonies were sequenced in the 1.5 population (1.5.1=SEQ ID NO:19; 1.5.2=SEQ ID NO:20), where 1.5.1 was observed 5 times (indicated by “×5”) and 1.5.2 was observed 2 times (indicated by “×2”). Amino acid diversity has decreased and started to show dominant clones present in final enriched B7-H3-binding affibody population.

FIG. 4. ABY 1.5.2 (SEQ ID NO:20) was genetically combined to GSG₃SG₃KG₃GT (GSGGGSGGGKGGGGT) (SEQ ID NO:35) linker and SortaseA₆₀₋₂₀₆ and produced in bacteria. ABY 1.5.2-L₁₅-SrtA had a K_{d} of 4.9 nM (4.0-5.9 nM). Error bars show standard error, with at least two replicates for each concentration.

FIG. 5 shows the sequences of the top performers after mutation of 1.5 population and then sorting an avidity sort, affinity sort, specificity sort, and stability sort. Sequence space shows variability compared to affibody 1.5.1 of the 1.5 population (see FIG. 3), with the exception of Otu8 and Otu394, which are point mutants of 1.5.1.

FIG. 6 shows the relative enrichment at each sorting step, as well as the weighted average z-score, for the top variants (sequences are shown in FIG. 5). Values greater than 1 indicate enrichment; values less than 1 indicate depletion. The color bars for affinity enrichment range from 1−M+2SD, as the mean (M) was slightly less than 1. Specificity, stability, and frequency color bars are displayed as M−M+2SD. The z-score is displayed 1-10.

FIG. 7 is a plot showing the relative frequency of top affibodies after each sort (avidity sort, affinity sort, specificity sort, and stability sort). Each line represents a single variant. Select variants are labeled with sequence name from FIG. 5.

### DETAILED DESCRIPTION OF ILLUSTRATIVE EMBODIMENTS

This disclosure describes anti-B7-H3 affibodies, compounds that include at least one of the anti-B7-H3 affibodies, and methods of using such compounds. As used herein, the term “anti-B7-H3 compound” refers to a compound that includes, or is, an anti-B7-H3 affibody. Exemplary platforms in which an anti-B7-H3 compound may be used include, but are not limited to, chimeric antigen receptor therapies (e.g., CAR-NK therapy, CAR-T therapy, CAR-macrophage therapy, etc.), multispecific immune cell engager technologies (e.g., bispecific killer engagers, trispecific killer engagers, bispecific T cell engagers, trispecific T cell engagers, etc.), targeted immunotherapies (e.g., targeted ADAM17 blocker (TAB) therapy), delivery of therapeutics (e.g., antibody-drug conjugates, delivery of therapeutic radioisotopes, delivery of toxins, delivery of cytokines, delivery of chemokines), imaging technologies (delivery of labeling constructs and/or labeling radioisotopes), or cell and/or ligand capture technologies (e.g., ELISA, etc.).

B7 Homolog 3 (B7-H3), also known as cluster of differentiation 276 (CD276), is a human protein encoded by the CD276 gene. The B7-H3 protein is a 316 amino acid-long type I transmembrane protein existing in two isoforms determined by its extracellular domain. B7-H3 mRNA is expressed in most normal tissues. In contrast, B7-H3 protein has a very limited expression on normal tissues because of its post-transcriptional regulation by microRNAs. In normal tissues, B7-H3 has a predominantly inhibitory role in adaptive immunity, suppressing T cell activation and proliferation. As such, B7-H3 is an important immune checkpoint inhibitor of T-cell function, is a tumor vasculature biomarker, and is overexpressed in a variety of cancers, including clear cell renal cell carcinoma, cutaneous melanoma, diffuse intrinsic pontine glioma, hypopharyngeal squamous cell carcinoma, non-small cell lung cancer, ovarian cancer, prostate cancer, and pancreatic cancer. The expression of B7-H3 is associated with tumor growth and metastasis and may ultimately lead to poor clinical prognosis.

Molecules that recognize certain targets specifically and with high affinity are useful for many clinical (e.g., diagnostic and/or therapeutic) and biotechnology applications. Typically, antibodies have been used for many of these applications, but antibodies have certain properties that may be drawbacks in certain applications. The limitations of antibodies have encouraged investigation toward alternative protein scaffolds that allow one to efficiently generate improved binding molecules. In the context of targeting solid tumors, for example, antibodies-which are typically about 150 kDa for immunoglobulin G (IgG)-can exhibit, due at least in part to their size, poor extravasation from vasculature, poor penetration through tissue, and/or long plasma clearance halftime, which can lead to poor signal-to-noise ratio, especially for diagnostic imaging. Antibodies also can exhibit thermal instability, which can lead to a loss of efficacy as a result of denaturation and/or aggregation. In addition, antibodies are typically made in mammalian cultures because many possess disulfide bonds, glycosylation, and/or multi-domain structures. This intricate structure can interfere with engineering the antibody for a particular application such as, for example, production of protein fusions for multispecific formats. Moreover, the presence of disulfide bonds in antibody molecules often precludes their intracellular use.

As a result of the limitations inherent to antibodies, alternative protein scaffolds have been developed in attempts to address many or all of these shortcomings. This disclosure describes recombinant, non-naturally occurring protein scaffolds, termed affibodies, capable of binding to the extracellular domain of B7-H3. In particular, the affibodies described herein may be used to display defined helical surfaces that are analogous to the complementarity-determining regions (“CDRs”) of an antibody variable region. The affibodies may be assembled into a multispecific compound capable of binding the extracellular domain of B7-H3 and one or more different targets. The affibodies described herein may therefore provide functional properties typically associated with antibody molecules. In particular, despite the fact that the affibody scaffold is not an immunoglobulin, it is similar in relevant respect to that of the variable region of the IgG heavy chain, making it possible for a protein scaffold to display an engineered binding paratope analogous to antibody CDRs. Because of this structure, the affibodies described herein possess ligand binding properties that may be similar in nature and affinity to the binding properties of antigen and antibody.

In one aspect, this disclosure describes anti-B7-H3 compounds. The anti-B7-H3 compounds are or include an anti-B7-H3 affibody. As used herein, the term “affibody” refers to a protein that includes at least one antigen binding site that binds to a particular antigen of interest. The anti-B7-H3 affibodies of the present disclosure bind to the extracellular domain of the B7-H3 protein, hereinafter B7-H3. The anti-B7-H3 affibodies of the present disclosure are developed from the affibody of SEQ ID NO:1.

The anti-B7-H3 affibodies of the present disclosure include the amino acid sequence AEAKYXKEXXXAXXXIXXLPNLXXXQXXAFXXXLXXDPSQSSELXXEAXKLNDSQAP K (SEQ ID NO:2), or a structurally similar sequence. Each X in SEQ ID NO:2 is a natural amino acid, an unnatural amino acid, or a modified amino acid, independent of the amino acid residues at any other positions within the affibody. In one or more embodiments, X at position 6 is Y, N, S, T, D, or A; X at position 9 is L, R, K, W, D, I, N, or Q; X at position 10 is H, A, S, F, I, D, E, Q, T, N, or L; X at position 11 is T, Y, D, A, L, P, I, or N; X at position 13 is I, Y, L, V, T, or F; X at position 14 is Q, Y, L, V, G, F, E, A, M, or S; X at position 15 is V, S, Q, or E; X at position 17 is H, A, V, G, R, I, F, or L; X at position 18 is S, Y, L, G, T, N, M, D, R, W, E, or H; X at position 23 is T or A; X at position 24 is L, K, A, G, R, H, W, V, or E; X at position 25 is D, P, T, N, V, S, H, L, R, A, W, or E; X at position 27 is R, K, T, I, or A; X at position 28 is A, Y, R, N, D, Q, I, H, L, S, or F; X at position 31 is L, K, I, or T; X at position 32 is L, G, Y, R, A, F, V, S, or Q; X at position 32 is A or K; X at position 35 is F, S, V, H, Y, D, E, or K; X at position 36 is N or D; X at position 44 is L or F; X at position 46 is S or P; and X at position 49 is R or K.

As used herein, a polypeptide (e.g., an affibody) is “structurally similar” to a reference polypeptide (e.g., anti-B7-H3 affibody including SEQ ID NO:3) if the amino acid sequence of the polypeptide possesses a specified amount of identity compared to the reference polypeptide. Structural similarity of two polypeptides can be determined by aligning the residues of the two polypeptides (for example, a candidate polypeptide and the polypeptide of, for example, SEQ ID NO: 3) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate polypeptide is the polypeptide being compared to the reference polypeptide (e.g., SEQ ID NO:3). A candidate polypeptide can be isolated, for example, from an animal or other natural source, or can be produced using recombinant techniques, or chemically or enzymatically synthesized.

A pair-wise comparison analysis of amino acid sequences can be carried out using the BESTFIT algorithm in the GCG package (version 10.2, Madison WI). Alternatively, polypeptides may be compared using the Blastp program of the BLAST 2 search algorithm, as described by Tatiana et al., (FEMS Microbiol Lett, 174, 247-250 (1999)), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in a polypeptide may be selected from other members of the class to which the amino acid belongs. For example, it is well-known in the art of protein biochemistry that an amino acid belonging to a grouping of amino acids having a particular size or characteristic (such as charge, hydrophobicity, and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH₂. Likewise, biologically active analogs of a polypeptide containing deletions or additions of one or more contiguous or noncontiguous amino acids that do not eliminate a functional activity of the polypeptide are also contemplated.

An anti-B7-H3 affibody as described herein may have at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:2 where each X is independently selected from any amino acid. An anti-B7-H3 affibody as described herein may have at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:2 where X at position 6 is Y, N, S, T, D, or A; X at position 9 is L, R, K, W, D, I, N, or Q; X at position 10 is H, A, S, F, I, D, E, Q, T, N, or L; X at position 11 is T, Y, D, A, L, P, I, or N; X at position 13 is I, Y, L, V, T, or F; X at position 14 is Q, Y, L, V, G, F, E, A, M, or S; X at position 15 is V, S, Q, or E; X at position 17 is H, A, V, G, R, I, F, or L; X at position 18 is S, Y, L, G, T, N, M, D, R, W, E, or H; X at position 23 is T or A; X at position 24 is L, K, A, G, R, H, W, V, or E; X at position 25 is D, P, T, N, V, S, H, L, R, A, W, or E; X at position 27 is R, K, T, I, or A; X at position 28 is A, Y, R, N, D, Q, I, H, L, S, or F; X at position 31 is L, K, I, or T; X at position 32 is L, G, Y, R, A, F, V, S, or Q; X at position 32 is A or K; X at position 35 is F, S, V, H, Y, D, E, or K; X at position 36 is N or D; X at position 44 is L or F; X at position 46 is S or P; and X at position 49 is R or K.

An anti-B7-H3 affibody as described herein, may have at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO:3 through SEQ ID NO:20 or SEQ ID NO: 22 through SEQ ID NO:34.

An anti-B7-H3 affibody as describe herein may have at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to any one of SEQ ID NO:3 through SEQ ID NO:20 or SEQ ID NO: 22 through SEQ ID NO:34.

This disclosure does not describe a SEQ ID NO:21. Thus, any stated range of SEQ ID NOs that encompasses SEQ ID NO: 21 does not include SEQ ID NO:21. For example, the statement “any one of SEQ ID NO:2 through SEQ ID NO:34” refers to any one of SEQ ID NO:3 through SEQ ID NO:20 and any one of SEQ ID NO: 22 through SEQ ID NO: 34.

In one or more embodiments, amino acids in the helical surface regions in any one of SEQ ID NO:2 through SEQ ID NO:34 may be mutated. The mutation may include substitution of one or more amino acid residues with a different amino acid residue, deletion of one or more amino acid residues, addition of one or more amino acid residues, or any combination thereof of any one of SEQ ID NO:2 through SEQ ID NO:34.

For example, in one or more embodiments, there may be one or more mutations in amino acid residues 6-18 of any one of SEQ ID NO:2 through SEQ ID NO:34. In some of those embodiments, residue 6 of any one of SEQ ID NO:2 through SEQ ID NO:34 may be mutated. In one or more embodiments, there may be one or more mutations in amino acid residues 9-11 of any one of SEQ ID NO:2 through SEQ ID NO:34. In one or more embodiments, there may be one or more mutations in amino acid residues 13-15 of any one of SEQ ID NO:2 through SEQ ID NO: 34. In one or more embodiments, amino acid residues 17 and/or 18 of any one of SEQ ID NO: 2 through SEQ ID NO:34 may be mutated. In one or more embodiments, there may be one or more mutations in amino acid residues 24-36 of SEQ ID NO:19. In some of those embodiments, residue 24 and/or 25 of any one of SEQ ID NO:2 through SEQ ID NO:34 may be mutated. In one or more embodiments, residue 27 and/or 28 of any one of SEQ ID NO:2 through SEQ ID NO:34 may be mutated. In one or more embodiments, there may be one or more mutations in amino acid residues 31-33 of any one of SEQ ID NO:2 through SEQ ID NO:34. In one or more embodiments, residues 35 and/or 36 of any one of SEQ ID NO:2 through SEQ ID NO: 34 may be mutated. In one or more embodiments, there may be one or more mutations in any of the amino acid residue regions described herein.

In one or more embodiments, there may be one or more amino acid mutations outside the helical surface regions of any one of SEQ ID NO:2 through SEQ ID NO:34. For example, in one or more embodiments, there may be one or more mutations in amino acid residues 1-5 of any one of SEQ ID NO:2 through SEQ ID NO:34. In one or more embodiments, there may be one or more mutations in amino acid residues 19-23 of any one of SEQ ID NO:2 through SEQ ID NO: 34. In one or more embodiments, there may be one or more mutations in amino acid residues 37-58 of any one of SEQ ID NO:2 through SEQ ID NO:34.

In one or more embodiments, there may be one or more mutations in one or more helical surface regions and one or more amino acid mutations outside the helical surface regions of any one of SEQ ID NO:2 through SEQ ID NO:34.

Table 1 shows the amino acid sequences of the parental affibody (SEQ ID NO:1) from which the anti-B7-H3 affibodies of the present disclosure are derived; a general consensus sequence (SEQ ID NO:2); a second consensus sequence (SEQ ID NO:3) for SEQ ID NO:4 through SEQ ID NO:18. Table 1 also shows example identities of X for the general consensus sequence (SEQ ID NO:2) as well as example mutations for SEQ ID NO:4 or any one of SEQ ID NO: 3 through SEQ ID NO:34. In one or more embodiments, the anti-B7-H3 affibody scaffolds of the present disclosure include any one of SEQ ID NO:2 through SEQ ID NO:34 having any combination of example possible mutations or possible identities of X shown in Table 1.

TABLE 1

| | SEQ | SEQ | SEQ | Example possible mutations for any one of | | | | | | | | | | | |
|---|---|---|---|---|---|---|---|---|---|---|---|---|---|---|---|
| Residue | ID | ID | ID | SEQ ID NO: 3 through SEQ ID NO: 34 and | | | | | | | | | | | |
| Number | NO: 1 | NO: 2 | NO: 3 | possible identities of X for SEQ ID NO: 2 | | | | | | | | | | | |
| 1 | A | A | A | | | | | | | | | | | | |
| 2 | E | E | E | | | | | | | | | | | | |
| 3 | A | A | A | | | | | | | | | | | | |
| 4 | K | K | K | | | | | | | | | | | | |
| 5 | Y | Y | Y | | | | | | | | | | | | |
| 6 | A | X | Y | Y | N | S | T | D | A | | | | | | |
| 7 | K | K | K | | | | | | | | | | | | |
| 8 | E | E | E | | | | | | | | | | | | |
| 9 | Q | X | L | L | R | K | W | D | I | N | Q | | | | |
| 10 | Q | X | H | H | A | S | F | I | D | E | Q | T | N | L | |
| 11 | N | X | N | T | Y | D | A | Y | L | P | I | N | | | |
| 12 | A | A | A | | | | | | | | | | | | |
| 13 | F | X | I | I | Y | L | V | T | F | | | | | | |
| 14 | Y | X | G | Q | Y | L | V | G | F | E | A | M | S | | |
| 15 | E | X | V | V | S | Q | E | | | | | | | | |
| 16 | I | I | I | | | | | | | | | | | | |
| 17 | L | X | R | H | A | V | G | R | I | F | L | | | | |
| 18 | H | X | S | Y | L | G | S | T | N | M | D | R | W | E | H |
| 19 | L | L | L | | | | | | | | | | | | |
| 20 | P | P | P | | | | | | | | | | | | |
| 21 | N | N | N | | | | | | | | | | | | |
| 22 | L | L | L | | | | | | | | | | | | |
| 23 | T | X | T | T | A | | | | | | | | | | |
| 24 | E | X | R | L | K | A | G | R | H | W | V | E | | | |
| 25 | E | X | D | D | P | T | N | V | S | H | L | R | A | W | E |
| 26 | Q | Q | Q | | | | | | | | | | | | |
| 27 | R | X | R | K | T | I | A | R | | | | | | | |
| 28 | N | X | A | Y | A | R | N | D | Q | I | H | A | L | S | F |
| 29 | A | A | A | | | | | | | | | | | | |
| 30 | F | F | F | | | | | | | | | | | | |
| 31 | I | X | L | K | L | I | T | | | | | | | | |
| 32 | Q | X | L | L | G | Y | R | A | F | V | S | Q | | | |
| 33 | K | X | A | A | K | | | | | | | | | | |
| 34 | L | L | L | | | | | | | | | | | | |
| 35 | K | X | F | F | S | V | H | Y | D | E | K | | | | |
| 36 | D | X | N | N | D | | | | | | | | | | |
| 37 | D | D | D | | | | | | | | | | | | |
| 38 | P | P | P | | | | | | | | | | | | |
| 39 | S | S | S | | | | | | | | | | | | |
| 40 | Q | Q | Q | | | | | | | | | | | | |
| 41 | S | S | S | | | | | | | | | | | | |
| 42 | S | S | S | | | | | | | | | | | | |
| 43 | E | E | E | | | | | | | | | | | | |
| 44 | L | L | L | | | | | | | | | | | | |
| 45 | L | X | L | F | L | | | | | | | | | | |
| 46 | S | X | S | P | S | | | | | | | | | | |
| 47 | E | E | E | | | | | | | | | | | | |
| 48 | A | A | A | | | | | | | | | | | | |
| 49 | K | X | K | R | K | | | | | | | | | | |
| 50 | K | K | K | | | | | | | | | | | | |
| 51 | L | L | L | | | | | | | | | | | | |
| 52 | N | N | N | | | | | | | | | | | | |
| 53 | D | D | D | | | | | | | | | | | | |
| 54 | S | S | S | | | | | | | | | | | | |
| 55 | Q | Q | Q | | | | | | | | | | | | |
| 56 | A | A | A | | | | | | | | | | | | |
| 57 | P | P | P | | | | | | | | | | | | |
| 58 | K | K | K | | | | | | | | | | | | |

Variants of the disclosed sequences also include affibody fragments, or full-length affibodies, that contain substitutions, deletions, or insertions into the protein backbone, that would still leave at least about 70% homology to the original affibody over the corresponding portion. A yet greater degree of departure from homology is allowed if like-amino acids, i.e., conservative amino acid substitutions, do not count as a change in the sequence. Examples of conservative substitutions involve amino acids that have the same or similar properties. Illustrative amino acid conservative substitutions include the changes of: alanine to serine; arginine to lysine; asparagine to glutamine or histidine; aspartate to glutamate or asparagine; cysteine to serine; glutamine to asparagine or glutamate; glutamate to aspartate or glutamine; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine or alanine; threonine to serine; tryptophan to tyrosine; tyrosine to tryptophan or phenylalanine; valine to isoleucine or leucine.

In one or more embodiments, an anti-B7-H3 affibody of the present disclosure may include additional sequences, such as, for example, amino acids appended to the C-terminal or N-terminal of the anti-B7-H3 affibody. Such modifications can, for example, facilitate purification by trapping on columns, the use of antibodies, or facilitate recovery when expressed recombinantly in a microbe. Such tags include, for example, a histidine-rich tag that allows purification of proteins on nickel columns and/or a leader sequence that can traffic a recombinantly-expressed anti-B7-H3 affibody to the membrane of the cell in which it is recombinantly expressed. Such gene modification techniques and suitable additional sequences are well known in the molecular biology arts. In one or more embodiments, the C-terminal and/or N-terminal modification may be cleaved from the anti-B7-H3 affibody before being incorporated into, for example, a pharmaceutical composition. In other embodiments, retaining a C-terminal or N-terminal modification may be desired for a given application—e.g., to facilitate immobilization to a substrate.

In one or more embodiments, an anti-B7-H3 affibody described herein also may include N-terminal or C-terminal functionalities other than a carboxylic acid or free amine. For example, the C-terminus, N-terminus, or both, of an affibody may be acylated, for example, acetylated. In one or more embodiments, the functionality may include a polyol (e.g., polyethylene glycol).

In one or more embodiments, an anti-B7-H3 affibody described herein may include post-translational modifications on one or more amino acids. Examples of post-translational modifications include, but are not limited to, acetylation, methylation, glycosylation, phosphorylation, prenylation, sulfonation, palmitoylation, hydroxylation, nitration, myristoylation, formylation, or citrullination. Post-translational modifications may be incorporated specifically or non-specifically through methods known in the biological arts.

In one or more embodiments, the anti-B7-H3 affibodies may be produced recombinantly in a suitable host cell and then purified. Methods for recombinant production and purification of proteins are well known in the art.

In one or more embodiments, the anti-B7-H3 affibodies of the present disclosure may be produced using solid phase peptide synthesis. In this method of production, the anti-B7-H3 affibody may be synthesized as one complete polypeptide or in multiple polypeptide fragments that can be joined using native chemical ligation, expressed protein ligation, Staudinger ligation, or Ser/Thr ligation. Methods for solid phase peptide synthesis and ligation techniques are well known in the art.

In another aspect, this disclosure describes polynucleotides that encode any of the anti-B7-H3 affibodies described herein, and the complements of such polynucleotide sequences. Given the amino acid sequence of any of the anti-B7-H3 affibodies described herein, a person of ordinary skill in the art can determine the full scope of polynucleotides that encode that amino acid sequence using conventional, routine methods.

The anti-B7-H3 affibodies of the present disclosure may be engineered using various techniques and assays. For example, magnetic activated cell sorting (MACS) and fluorescence activated cell sorting (FACS) may be used to select affibodies of interest that bind to B7-H3 in yeast display model.

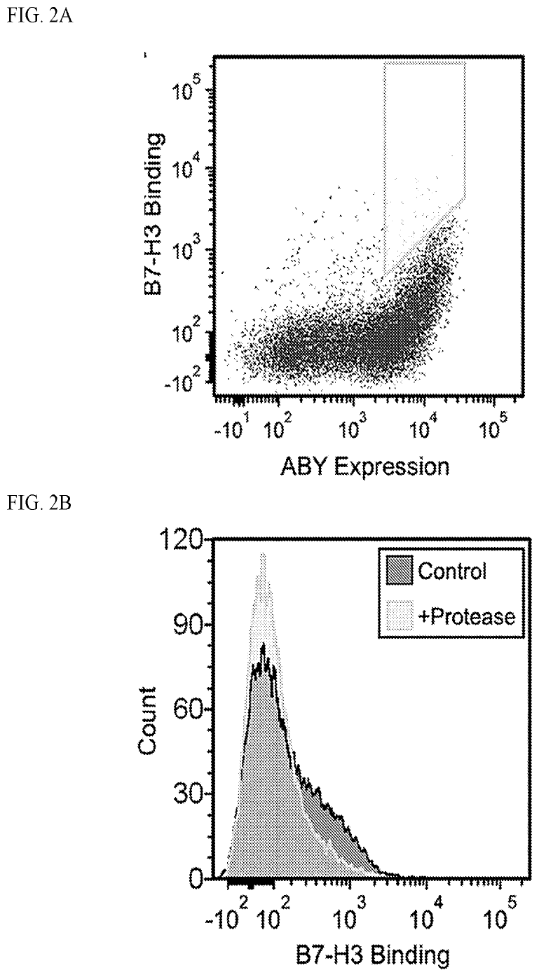

The following describes a campaign that was used to engineer at least a portion of the anti-B7-H3 affibodies of the present disclosure (see Example 1 and Example 2 for additional details). An affibody scaffold library that included affibody amino acid sequences derived from SEQ ID NO: 1 was used to discover binders to B7-H3 via yeast display, magnetic activated cell sorting, and fluorescence activated cell sorting. A library (0.0 affibody population, otherwise called a library) underwent three MACS selections (to give a 0.1, a 0.2, and a 0.3 affibody population) using recombinant human B7-H3 extracellular domain immobilized on magnetic beads, followed by a sole FACS selection using the same recombinant B7-H3 protein at 50 nM and gated to select for affibodies with moderate affinity resulting in a 0.4 population. DNA was isolated from the entire 0.4 affibody population, randomly mutated via error-prone PCR, and electroporated back into yeast. The mutated naïve population underwent a MACS selection (to give a 1.1 affibody population), followed by a monovalent MACS sort (to give a 1.2 affibody population) with 100 nM recombinant human B7-H3. The 1.2 affibody population was then further enriched with two FACS selections (to give a 1.3 and 1.4 affibody population) with detergent solubilized MS1-B7-H3 lysate of increasing stringency, using estimated B7-H3 lysate concentrations of 50 nM and 1 nM. Yeast were grown and reinduced between each selection. The 1.4 affibody population continued to another round of selection to sort for protease and thermostable affibodies with B7-H3 binding resulting in population 1.5 (FIG. 2).

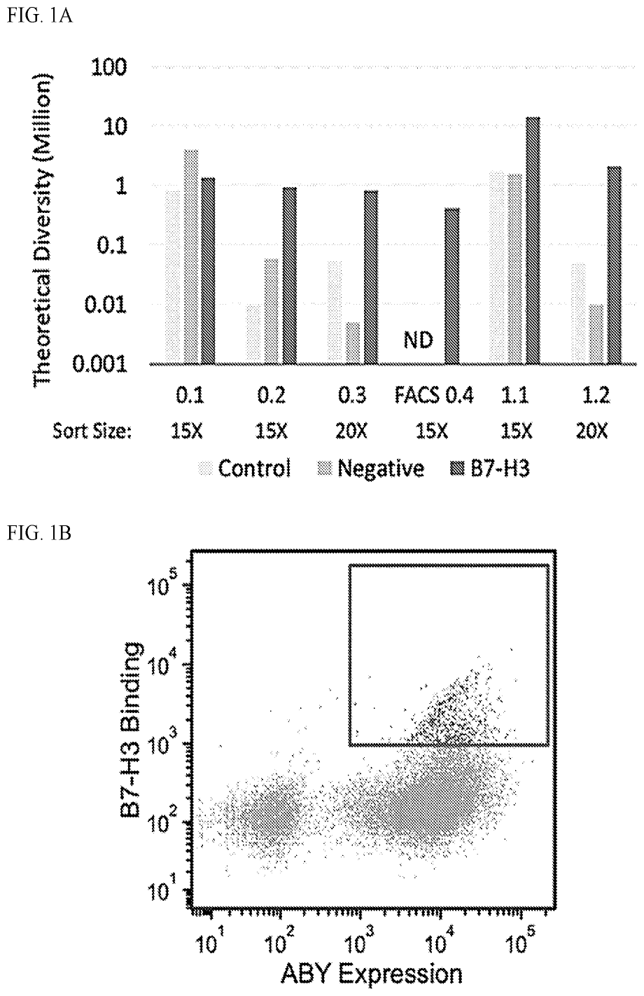

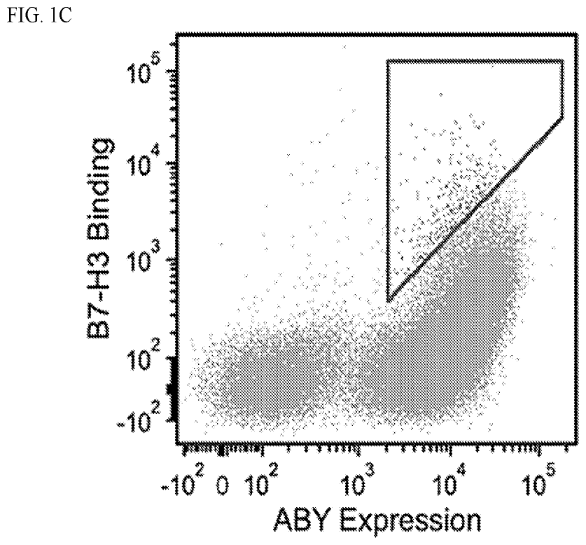

FIG. 1A is a plot showing the theoretical diversity of affibody populations after various sorts (0.4 is the 0.4 population). Populations 0.2, 0.3, and 1.2 exhibited specific binding to B7-H3 and not the negative control. The higher the theoretical diversity, the more cells were collected for the stated parameter (e.g., control, negative, or B7-H3 binder). Population 1.1 had the highest theoretical diversity of cells that bound B7-H3. FIG. 1B is a flow cytometric analysis (FACS) of 10,000 random variants of the 0.4 population. This resulted in moderate affinity binders to recombinant B7-H3. FIG. 1C is a flow cytometric analysis of the enriched 1.4 population that resulted from mutation of the 0.4 population in FIG. 1B (shown in gray box) and subsequent sorts, as specified previously. FIG. 1C was labeled with approximately 1 nM biotin-B7-H3 cell lysate, streptavidin-AF647, and chicken anti-c-Myc-FITC. This resulted in high affinity binders to cellular B7-H3. The boxes in FIG. 1B and FIG. 1C show the population that was collected during the FACS sort. The collected cells are expressed full length affibody and bound to B7-H3. A rectangle was drawn in FIG. 1B to selected for binders, as a majority of the population was non-binding. In FIG. 1C, a diagonal polygon was drawn to be more stringent and collect for top binders with higher affinity.

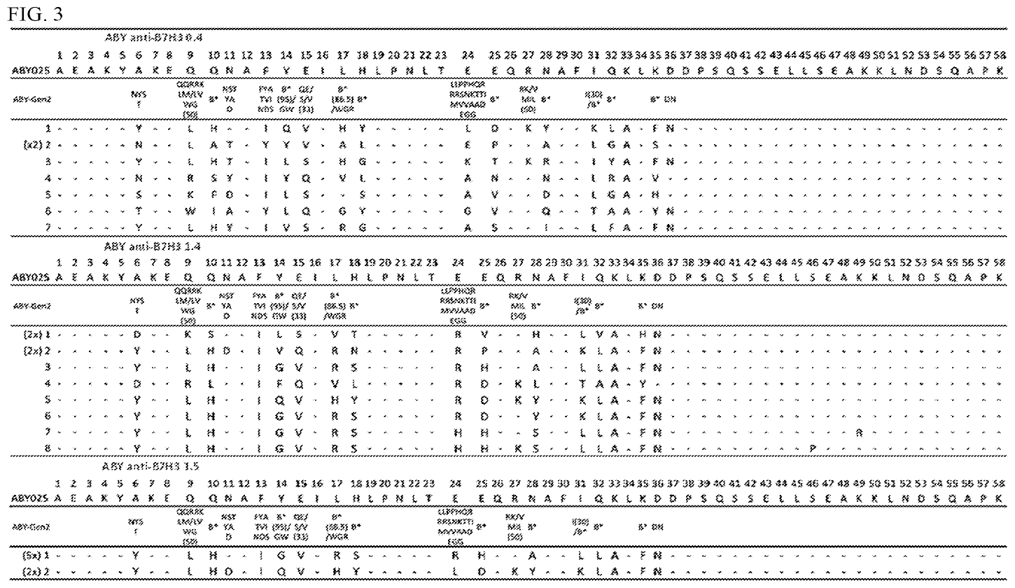

Single colonies of affibody populations were stochastically chosen and Sanger sequenced at two selection points (see FIG. 3): (1) after the first FACS sort (fourth sort overall; resulting in the 0.4 population), as well as after additional selections (two MACS selections and two FACS lysate selections; resulting in the 1.4 population) post-error-prone PCR. Affibody variants were Sanger sequenced a third time following the protease and thermostability B7-H3 binding sort (resulting in the 1.5 population). Sequencing results for affibody are shown in FIG. 3 (ABY025 is the affibody sequence that the anti-B7-H3 affibodies were derived from =SEQ ID NO:1; 0.4.1=SEQ ID NO:4; 0.4.2=SEQ ID NO:5; 0.4.3=SEQ ID NO:6; 0.4.4=SEQ ID NO: 7; 0.4.5=SEQ ID NO:8; 0.4.6=SEQ ID NO:9; 0.4.7=SEQ ID NO: 10; 1.4.1=SEQ ID NO: 11; 1.4.2=SEQ ID NO:12; 1.4.3=SEQ ID NO:13; 1.4.4=SEQ ID NO:14; 1.4.5=SEQ ID NO:15; 1.4.6=SEQ ID NO: 16; 1.4.7=SEQ ID NO: 17; 1.4.8=SEQ ID NO: 18; 1.5.1=SEQ ID NO: 19; 1.5.2=SEQ ID NO20).

The 1.5 affibody population was further engineered, specificity for affinity, specificity, and stability maturation (see Example 2 for additional details). DNA was isolated from the 1.5 yeast population and subjected to random mutagenesis via error-prone polymerase chain reaction (PCR) of the variable regions of the affibodies. DNA was electroporated back into yeast for display and further sorted using the following sort scheme: (1) lysate MACS with 1 pmol B7-H3+ lysate sort (avidity sort); (2) an affinity sort (strict K_{d} FACS sort); (3) specificity sort (multitarget MACS depletion sort); and (4) stability sort (thermolysin at 55° C. FACS sort). After each sort, the populations were deep sequenced.

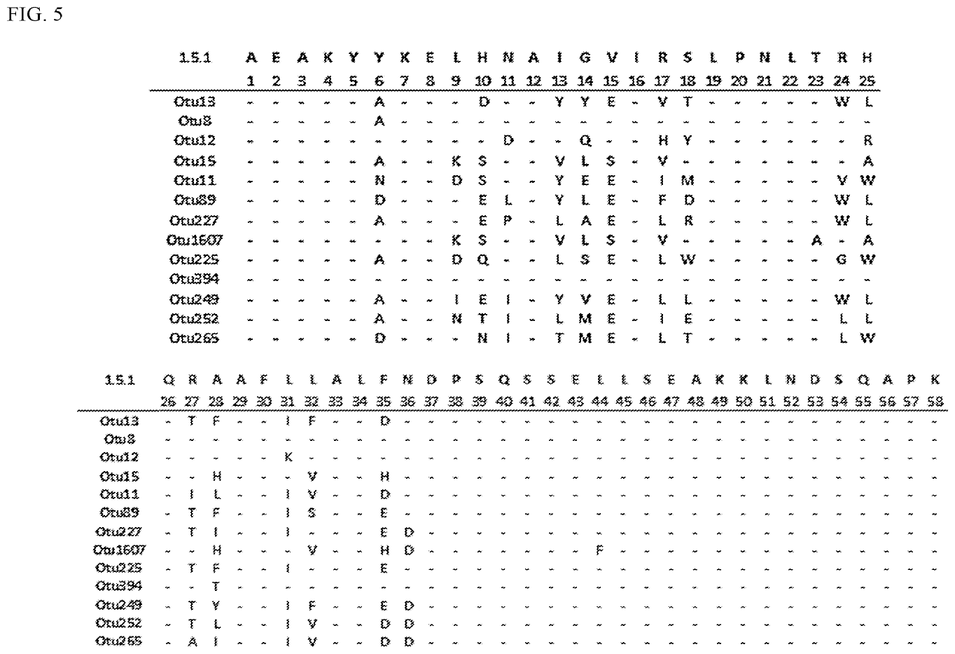

The frequencies at each sort (e.g., avidity, affinity, specificity, and stability) were determined for each sequence, as well as their subsequent enrichment. If the read count was 0, a read count of 1 was assigned. The z-scores for affinity, specificity, stability, and final frequency of the top variant was determined and a final conditional, weighted averaged z-score was calculated (see Example 2). Top performers were selected based on the highest score. FIG. 5 shows the sequences of the top performers of the 1.5.1 population after all sorts were completed.

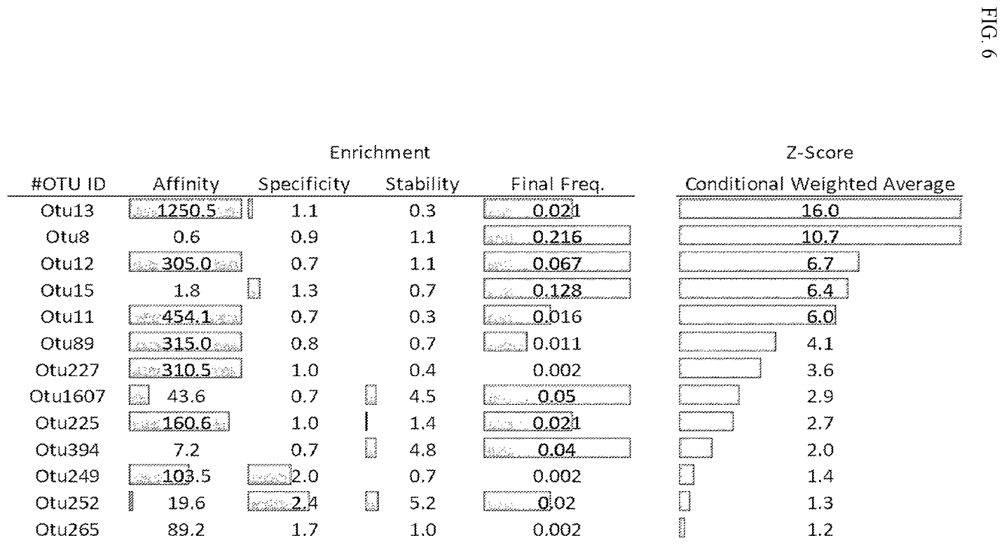

FIG. 6 shows the z-scores of affinity, specificity, stability, and final frequency as well as the conditional weighted average z-score for the top variants. Values greater than 1 indicate enrichment and values less than 1 indicate depletion. In FIG. 6, the color bars for affinity range M−M+2SD enrichment, while specificity and stability are displayed 1−M+2SD, as the mean (M) was slightly less than 1. On the bar chart, the z-score is displayed 1-10 (i.e., if the z-score is 10 or above, the bar spans the entire length of the chart).

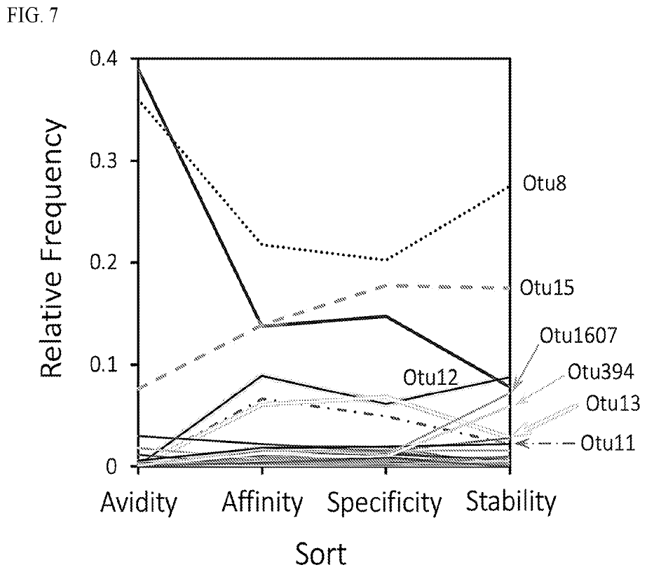

FIG. 7 is a plot showing the relative frequency of specific variants (each line) after each sort (avidity, affinity, specificity, and stability). An ideal variant would be indicated by a straight line at a high relative frequency across all sorts.

In addition to the anti-B7-H3 affibody, in one or more embodiments, the anti-B7-H3 compound may include a first functional component operably linked to the anti-B7-H3 affibody. In one or more embodiments, the first functional component may include a targeting component. In one or more embodiments, the first functional component may include an imaging component. In one or more embodiments, the first functional component may include an enzyme. In one or more embodiments, the first functional component may include a small molecule drug, a pharmacologically active derivative thereof, or an activatable inactive form of a drug (e.g., a prodrug).

In one or more embodiments, the first functional component includes a targeting component. In embodiments where the first functional component includes a targeting component, the anti-B7-H3 compound may be an immunotherapeutic compound. Immunotherapeutic compounds can provide individualized treatment that activates or suppresses the immune system to amplify or diminish an immune response and is developing rapidly for treating various forms of cancer. Immunotherapy for cancer, such as chimeric antigen receptor (CAR)-T cells, CAR-natural killer (NK) cells, PD-1 and PD-L1 inhibitors, aims to help a subject's immune system fight cancer.

In one or more embodiments, the targeting component may bind to and recruit an effector cell to the B7-H3 displaying cell. Types of effector cells include, but are not limited to, natural killer (NK) cells, B cells, or T cells. The targeting component may bind to a surface protein displayed on the effector cell. Non-limiting examples of surface proteins displayed on NK cells include, CD16, PD-1, NKp30, NKp40, NKp44, NKp46, NKG2C, or KIRs. Non-limiting examples of surface proteins displayed on B cells include CD4, CD8, or VLA-4. Non-limiting examples of surface proteins displayed on T cells include (LFA)-1, CD2, CD4, or CD8.

In one or more embodiments, the targeting component may include an antibody or fragment thereof, an affibody, a peptide, a protein, or a small molecule.

In one or more embodiments, the first functional component includes an imaging component. In embodiments where the first functional component includes an imaging component, the anti-B7-H3 compound may be used as a diagnostic tool to detect B7-H3 positive tumors.

The imaging component may be any component that can produce a detectable signal. Exemplary types of imaging components include, but are not limited to a colorimetric label, a fluorescent label, a radioactive label, a magnetic label, or an enzymatic label. Depending on the type of imaging component, various methods may be used to detect the imaging component. Example detection methods include magnetic resonance imaging (MRI), positron emission tomography (PET), single photon emission computed tomography (SPECT), ultrasound, photoacoustic imaging, fluorescence microscopy, total internal reflection fluorescence (TIRF)-microscopy, or stimulated emission depletion (STRED)-nanoscopic. The imaging techniques may be accomplished in vivo or in vitro.

In one or more embodiments, the first functional component may include an enzyme. In one or more embodiments, the enzyme may modify one or more of the surface proteins displayed on the cell displaying B7-H3 to which the anti-B7-H3 affibody is bound. Examples of enzymes that modifies surface proteins, include but are not limited to, a protease, a lipase, or a sortase such as sortase A.

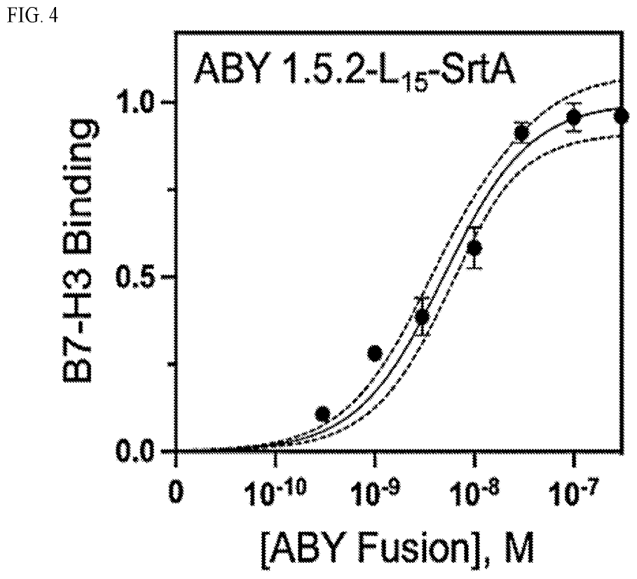

For example, to analyze the modularity of affibodies, affibody 1.5.2 (SEQ ID NO:20) were genetically linked to sortase A protein enzyme via a 15 amino acid glycine rich linker (SEQ ID NO: 35) at the affibody's C-terminus, i.e., affibody 1.5.2-L₁₅-SrtA fusion (see Example 1). Modularity was assessed by the retention of B7-H3 binding affinity of the affibody in the presence of glycine-linked sortase A enzyme. Although affibody 1.5.2 alone was not assessed for binding affinity, affibody 1.5.2-L₁₅-SrtA had a K_{d} of 4.9 nM (4.0-5.9 nM), demonstrating low-nanomolar affinity in the presence of linked sortase A (FIG. 4).

In one or more embodiments, the first functional component may include a small molecule drug. As used herein, the term “drug” is used to collectively refer to a pharmacologically active substance or an activatable inactive form of a pharmacologically active substance (e.g., a prodrug). In embodiments where the first functional component includes a small molecule drug, the anti-B7-H3 compound may be a chemotherapeutic compound. In one or more embodiments, the chemotherapeutic compound may include one or more radioisotopes. In embodiments where the chemotherapeutic compound includes one or more radioisotopes, the anti-B7-H3 compound may be used for radioisotope therapy (radionuclide therapy). Examples of suitable isotopes include iodine-131, iridium-192, strontium-89, samarium-153, rhenium-186, boron-10, phosphorus-32, or radium 223. The anti-B7-H3 affibody may allow for localization of the small molecule drug to B7-H3 displaying cells, such as tumor cells, thereby delivering the chemotherapeutic compound to a tumor cell that expresses B7-H3. In this way, the chemotherapeutic compound may be administered to a subject systematically yet have increased local activity and reduced systemic side effects compared to administering the drug systemically without being a component of an anti-B7-H3 compound.

In one or more embodiments, the anti-B7-H3 compound may include additional functional components in addition to the first functional component and the anti-B7-H3 affibody. For example, the anti-B7-H3 compound may include a second functional component, a third functional component, a fourth functional component, a fifth functional component, a sixth functional component, or a seventh functional component. Each functional component may include an anti-B7-H3 affibody or include an imaging component, an enzyme, a small molecule drug, or a targeting component (all of which are previously described herein), independent of the identity of any other component in the anti-B7-H3 compound.

In some embodiments where the anti-B7-H3 compound includes at least one functional component, the anti-B7-H3 affibody and the functional component are operably linked. As used herein, the term “operably linked” refers to a direct or indirect covalent linking between the anti-B7-H3 affibody and any additional functional component or functional components of the anti-B7-H3 compound. Thus, two functional components, or one functional component and the anti-B7-H3 affibody, that are operably linked may be directly covalently coupled to one another. Conversely, two operably linked functional components, or one functional component operably linked to the anti-B7-H3 affibody, may be connected by mutual covalent linking to an intervening component (e.g., a flanking sequence or linker). For example, in embodiments where the anti-B7-H3 compound includes two functional components, the first functional component and the second functional component may be separately directly linked to the anti-B7-H3 affibody; or the first functional component may be directly linked to the anti-B7-H3 affibody and the second functional component directly linked to the first functional component.

The anti-B7-H3 affibody and the first functional component may be operably linked through one or more linkers. The term “linker” as used herein refers to any bond, small molecule, peptide sequence, or other vehicle that physically links the functional components of the anti-B7-H3 compound. Linkers can be susceptible to or be substantially resistant to acid-induced cleavage, light-induced cleavage, peptidase-induced cleavage, esterase-induced cleavage, and/or disulfide bond cleavage at conditions under which the first functional (or other functional components) component and/or the anti-B7-H3 affibody remains active. Linkers are classified based on the presence of one or more chemical motifs such as, for example, including a disulfide group, a hydrazine group or peptide (cleavable), or a thioester group (non-cleavable). Linkers also include charged linkers, and hydrophilic forms thereof as known in the art.

Suitable linkers for linking the anti-B7-H3 affibody and the first functional component of the anti-B7-H3 compounds of the present disclosure include a natural linker, an empirical linker, or a combination of natural and/or empirical linkers. Natural linkers are derived from the amino acid linking sequence of multi-domain proteins, which are naturally present between protein domains. Properties of natural linkers such as, for example, length, hydrophobicity, amino acid residues, and/or secondary structure can be exploited to confer desirable properties to a multi-domain compound that includes natural linkers connecting the components of the anti-B7-H3 compounds of the present disclosure.

The studies of linkers in natural multi-domain proteins have led to the generation of many empirical linkers with various sequences and conformations for the construction of recombinant fusion proteins. Empirical linkers are often classified as three types: flexible linkers, rigid linkers, and cleavable linkers. Flexible linkers can provide a certain degree of movement or interaction at the joined components. Flexible linkers typically include small, non-polar (e.g., Gly) or polar (e.g., Ser or Thr) amino acids, which provide flexibility, and allow for mobility of the connected components. An example of a flexible linker useful for linking the anti-B7-H3 affibody and the first functional component of the anti-B7-H3 compounds of the present disclosure is SEQ ID NO:35. Rigid linkers can successfully keep a fixed distance between the first functional component and the anti-B7-H3 affibody of the anti-B7-H3 compounds to maintain their independent functions, which can provide efficient separation of the first functional component and the anti-B7-H3 affibody and/or sufficiently reduce interference between the first functional component and the anti-B7-H3 affibody. Cleavable linkers can allow one to control the release of the first functional component and/or the anti-B7-H3 affibody in vivo. By taking advantage of unique in vivo processes, cleavable linkers can be cleaved under specific conditions such as the presence of reducing reagents or proteases. This type of linker can reduce steric hindrance, improve bioactivity, and/or achieve independent actions/metabolism of the first functional component and/or the anti-B7-H3 affibody after linker cleavage.

Exemplary suitable linker sequences include the amino acid sequences of SEQ ID NO: 35, SEQ ID NO:36, SEQ ID NO:37, SEQ ID NO:38, SEQ ID NO:39, SEQ ID NO:40, SEQ ID NO: 41, SEQ ID NO:42, SEQ ID NO:43, SEQ ID NO:44, SEQ ID NO:45, SEQ ID NO:46, and SEQ ID NO:47.

In one or more embodiments, the natural linker or empirical linker is covalently attached to the anti-B7-H3 affibody, the first functional component, or both, using bioconjugation chemistries. Bioconjugation chemistries are well known in the art and include, but are not limited to, NHS-ester ligation, isocyanate ligation, isothiocyanate ligation, benzoyl fluoride ligation, maleimide conjugation, iodoacetamide conjugation, 2-thiopyridine disulfide exchange, 3-arylpropiolonitrile conjugation, diazonium salt conjugation, 4-phenyl-3H-1,2,4-triazole-3,5 (4H)-dione conjugation, and Mannich ligation.

In one or more embodiments, the natural linker or empirical linker, the first functional component, the anti-B7-H3 affibody, or combinations thereof, may include one or more unnatural amino acids that allow for bioorthogonal conjugation reactions. As used herein, “bioorthogonal conjugation” refers to a conjugation reaction that uses one or more unnatural amino acids or modified amino acids as a starting reagent. Examples of bioorthogonal conjugation reactions include, but are not limited to, Staudinger ligation, copper-catalyzed azide-alkyne cycloaddition, strain promoted [3+2] cycloadditions, tetrazine ligation, metal-catalyzed coupling reactions, or oxime-hydrazone ligations. Examples of non-natural amino acids include, but are not limited to, azidohomoalanine, 2 homopropargylglycine, 3 homoallylglycine, 4 p-acetyl-Phe, 5 p-azido-Phe, 3-(6-acetylnaphthalen-2-ylamino)-2-aminopropanoic acid, N^{ε}-(cyclooct-2-yn-1-yloxy) carbonyl) L-lysine, N^{ε}-2-azideoethyloxycarbonyl-L-lysine, Nε-p-azidobenzyloxycarbonyl lysine, propargyl-L-lysine, or trans-cyclooct-2-ene lysine. Such amino acids may be incorporated at any location of the anti-B7-H3 affibodies of the present disclosure or at any location on any of the functional components.

In one or more embodiments, the linker is derived from a small molecule, such as a polymer. Example polymer linkers include but are not limited to, poly-ethylene glycol, poly(N-isopropylacrylamide), and N,N′-dimethylacrylamide)-co-4-phenylazophenyl acrylate. The small molecule linkers generally include one or more reactive handles allowing conjugation to the anti-B7-H3 affibody, the domain, or both. In one or more embodiments, the reactive handle allows for a bioconjugation or bioorthogonal conjugation. In one or more embodiments, the reactive handle allows for any organic reaction compatible with conjugating a linker to the anti-B7-H3 affibody or the first functional component.

The linker may be conjugated at any amino acid location of the anti-B7-H3 affibody. For example, the linker may be conjugated to the N-terminus, C-terminus, or any amino acid between.

In embodiments where the anti-B7-H3 compound includes more than one functional component, the additional functional components may be operably coupled to each other and/or the anti-B7-H3 affibody using one or more of the linkers disclosed herein.

In some embodiments where the anti-B7-H3 compound includes an anti-B7-H3 affibody and one or more functional components operably coupled by peptide linkers, the anti-B7-H3 compound may be produced by expression in a host cell using methods known in the art. In some embodiments where the anti-B7-H3 compound includes an anti-B7-H3 affibody and one or more functional components operably coupled by peptide linkers, the anti-B7-H3 compound may be produced using solid phase peptide synthesis using methods known in the art.

In another aspect, this disclosure describes an isolated nucleic acid sequence that encodes the amino acid sequence of any embodiment of the anti-B7-H3 compounds described herein or any component polypeptide fragment thereof. Given the amino acid sequence of any polypeptide, a person of ordinary skill in the art can determine the full scope of polynucleotides that encode that amino acid sequence using conventional, routine methods.

In another aspect, this disclosure describes a host cell including any of the isolated nucleic acid sequences, anti-B7-H3 compounds, and/or anti-B7-H3 affibodies described herein.

The nucleic acid constructs of the present disclosure may be introduced into a host cell to be altered, thus allowing expression of an anti-B7-H3 compound and/or an anti-B7-H3 affibody within the cell, thereby generating a genetically engineered cell. A variety of methods are known in the art and suitable for introduction of a nucleic acid into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (e.g., nanoparticles), cationic polymer mediated transfer (e.g., DEAE-dextran, polyethylenimine, polyethylene glycol (PEG) and the like), or cell fusion. Other methods of transfection include proprietary transfection reagents such as LIPOFECTAMINE (Thermo Fisher Scientific, Inc., Waltham, MA), HILYMAX (Dojindo Molecular Technologies, Inc., Rockville, MD), FUGENE (Promega Corp., Madison, WI), JETPEI (Polyplus Transfection, Illkirch, France), EFFECTENE (Qiagen, Hilden, Germany) and DreamFect (OZ Biosciences, Inc USA, San Diego, CA).

The nucleic acid constructs described herein may be introduced into a host cell to be altered, thus allowing expression within the cell of the anti-B7-H3 compound and/or the anti-B7-H3 affibody encoded by the nucleic acid. A variety of host cells are known in the art and suitable for protein expression. Examples of typical cell used for transfection and protein expression include, but are not limited to, a bacterial cell, a eukaryotic cell, a yeast cell, an insect cell, or a plant cell such as, for example, E. coli, Bacillus, Streptomyces, Pichia pastoris, Salmonella typhimurium, Drosophila S2, Spodoptera SJ9, CHO, COS (e.g., COS-7), 3T3-F442A, HeLa, HUVEC, HUAEC, NIH 3T3, Jurkat, 293, 293H, or 293F.

In another aspect, this disclosure provides pharmaceutical compositions that include one or more anti-B7-H3 compounds described herein (including anti-B7-H3 compounds that include an anti-B7-H3 affibody and no additional functional components), formulated together with a pharmaceutically acceptable carrier. Such compositions may include one or a combination of, for example, two or more different anti-B7-H3 compounds.

As used herein, “pharmaceutically acceptable” refers to a material that is not biologically or otherwise undesirable, e.g., the material may be administered to an individual along with an anti-B7-H3 compound without causing any undesirable biological effects or interacting in a deleterious manner with any of the other components of the pharmaceutical composition in which it is contained.

A pharmaceutical composition may include one or more pharmaceutically acceptable salts. Examples of such salts include acid addition salts and base addition salts. A pharmaceutical composition also, or alternatively, may include a pharmaceutically acceptable antioxidant. Those skilled in the art are generally aware of and understand how to apply the use of pharmaceutically acceptable salts and/or pharmaceutically acceptable antioxidants.

The pharmaceutical composition may be formulated with a pharmaceutically acceptable carrier. As used herein, the term “carrier” includes any solvent, dispersion medium, vehicle, coating, diluent, antibacterial, and/or antifungal agent, isotonic agent, absorption delaying agent, buffer, carrier solution, suspension, hydrogel, colloid, an accessory agent, stabilizer, protein carrier, biological carrier compound, or the like. Non-limiting examples of solvents include water, ethanol, a polyol (e.g., glycerol, propylene glycol, polyethylene glycol, and the like), a vegetable oil (e.g., olive oil), or an injectable organic ester (e.g., ethyl oleate), or combinations thereof. Non-limiting examples of a protein carrier includes keyhole limpet hemocyanin (KLH), bovine serum albumin (BSA), ovalbumin, or the like. Non-limiting examples of a biological compound which may serve as a carrier include a glycosaminoglycan, a proteoglycan, or albumin. The carrier may include an organic solvent (e.g., dimethyl sulfoxide), a synthetic compound, or a synthetic polymer (e.g., a polyalkyleneglycol). Ovalbumin, human serum albumin, other proteins, polyethylene glycol, or the like may be employed as the carrier. The use of such media and/or agents for pharmaceutical active substances is well known in the art. Except insofar as any conventional media or agent is incompatible with the active ingredient (i.e., the anti-B7-H3 compound), its use in the therapeutic compositions is contemplated. Supplementary active ingredients also can be incorporated into the compositions.

A pharmaceutical composition may also, or alternatively, may include one or more adjuvants such as, for example, a preservative, a wetting agent, an emulsifying agent, and/or a dispersing agent. In one or more embodiments, a pharmaceutical composition can include an antibacterial agent and/or an antifungal agent such as, for example, paraben, chlorobutanol, phenol sorbic acid, or the like. It may also be desirable to include isotonic agents, such as a sugar, sodium chloride, or a polyalcohol (e.g., mannitol, sorbitol, etc.) into the compositions. In addition, prolonged absorption of an injectable pharmaceutical form may be provided by including an agent that delays absorption such as, for example, aluminum monostearate or gelatin.

An anti-B7-H3 compound of the present disclosure may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release.

A formulation may be conveniently presented in unit dosage form and may be prepared by methods well known in the art of pharmacy. Methods of preparing a composition with a pharmaceutically acceptable carrier include the step of bringing the anti-B7-H3 compound into association with a carrier that constitutes one or more accessory ingredients. In general, a formulation may be prepared by uniformly and/or intimately bringing the active ingredient into association with a liquid carrier, a finely divided solid carrier, or both, and then, if necessary, shaping the product into the desired formulations.

To prepare pharmaceutical compositions including the anti-B7-H3 compounds of the present disclosure, the anti-B7-H3 compound can be mixed with a pharmaceutically acceptable carrier or excipient. Formulations of therapeutic and diagnostic agents can be prepared by mixing with physiologically acceptable carriers, excipients, or stabilizers in the form of, e.g., lyophilized powders, slurries, aqueous solutions, lotions, or suspensions.

Thus, an anti-B7-H3 compound of the present disclosure may be provided in any suitable form including, but not limited to, a solution, a suspension, an emulsion, a spray, an aerosol, or any form of mixture. The composition may be delivered in formulation with any pharmaceutically acceptable excipient, carrier, or vehicle.

Nasal spray formulations include purified aqueous solutions of the active ingredient with preservative agents and isotonic agents. Such formulations are preferably adjusted to a pH and isotonic state compatible with the nasal mucous membranes. Formulations for rectal or vaginal administration may be presented as a suppository with a suitable carrier such as cocoa butter, hydrogenated fats, or hydrogenated fatty carboxylic acids. Ophthalmic formulations are prepared by a similar method to the nasal spray, except that the pH and isotonic factors are preferably adjusted to match that of the eye.

Topical formulations include the anti-B7-H3 compound dissolved or suspended in one or more media such as mineral oil, petroleum, polyhydroxy alcohols, or other bases used for topical pharmaceutical formulations. For example, topical formulations may include a cream, an ointment, a paste, a lotion, a powder, a solid, an aerosolized foam, or a gel. Topical formulations may contain a permeation enhancer to increase the bioavailability of the active ingredient. Topical formulations may contain preservatives and/or emulsifiers. Topical formulations may be provided in the form of a transdermal patch or bandage, wherein the formulation is incorporated into a gauze or other structure and brought into contact with the skin. The formulation may further include one or more additives including such as, for example, an adjuvant, a skin penetration enhancer, a colorant, a fragrance, a flavoring, a moisturizer, a thickener, and the like.

Formulations of the present disclosure suitable for oral administration may be presented as discrete units such as a tablet, a troche, a capsule, a lozenge, a wafer, or a cachet, each containing a predetermined amount of the active ingredient as a powder or granules, as liposomes, or as a solution or suspension in an aqueous liquor or non-aqueous liquid such as a syrup, an elixir, an emulsion, or a draught. The tablet, troche, pill, capsule, and the like may also contain one or more of the following: a binder such as gum tragacanth, acacia, corn starch or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; or a natural or artificial flavoring agent.

When the unit dosage form is a capsule, it may further contain a liquid carrier, such as a vegetable oil or a polyethylene glycol. Various other materials may be present as coatings or to otherwise modify the physical form of the solid unit dosage form. For instance, a tablet, a pill, or a capsule may be coated with gelatin, wax, shellac, sugar, and/or the like. A syrup or elixir may contain one or more sweetening agent, preservative such as methyl- or propylparaben, an agent to retard crystallization of sugar, an agent to increase the solubility of any other ingredient (e.g., a polyhydric alcohol such as, glycerol or sorbitol), a dye, and/or flavoring agent. The material used in preparing any unit dosage form is substantially nontoxic in the amounts employed. The active ingredient may be incorporated into preparations or devices in formulations that may or may not be designed for sustained release.

Formulations suitable for parenteral administration can include a sterile aqueous preparation of the active ingredient, or a dispersion of a sterile powder of the active ingredient, which are preferably isotonic with the blood of the recipient. Parenteral administration of an anti-B7-H3 compound or pharmaceutical composition containing the same of the present disclosure (e.g., through an intravenous drip) is one form of administration. Isotonic agents that may be included in the liquid preparation include a sugar, a buffer, and/or sodium chloride. Solutions of the active ingredient may be prepared in water, optionally mixed with a nontoxic surfactant. Dispersions of the active ingredient may be prepared in water, ethanol, a polyol (such as glycerol, propylene glycol, liquid polyethylene glycols, and the like), a vegetable oil, a glycerol ester, or any mixtures thereof.

In yet another aspect, this disclosure provides imaging methods and methods of treating, ameliorating, detecting, diagnosing, or monitoring a disease or a symptom or clinical sign thereof, as described herein, in a patient by administering a therapeutically effective amount of an anti-B7-H3 compound described herein and/or a pharmaceutical composition that includes one or more anti-B7-H3 compounds described herein. As used herein, the term “treating” and variations thereof refer to reducing, limiting progression, ameliorating, or resolving, to any extent, the symptoms or clinical signs related to a condition. A “symptom” refers to any subjective evidence of disease or of a patient's condition; a “sign,” or “clinical sign” refers to an objective physical finding relating to a particular condition capable of being found by one other than the patient. A “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Generally, a “therapeutic” treatment is initiated after a condition manifests in a subject, while “prophylactic” treatment is initiated before a condition manifests in a subject. Prophylactic treatment may be administered to a subject at risk of having a condition. “At risk” refers to a subject that may or may not actually possess the described risk. In the case of a non-infectious condition, for example, a subject “at risk” for developing a specified condition is a subject that possesses one or more indicia of increased risk of having, or developing, the specified condition compared to individuals who lack the one or more indicia, regardless of the whether the subject manifests any symptom or clinical sign of having or developing the condition.

Thus, in one or more embodiments, treating a subject includes a subject having, or at risk of having cancer. Generally, the method includes administering to the subject an effective amount of an anti-B7-H3 compound. As used herein, the term “cancer” refers to a group of diseases characterized by abnormal and uncontrolled cell proliferation starting at one site (primary site) with the potential to invade and to spread to other sites (secondary sites, metastases) that differentiates cancer (malignant tumor) from benign tumor. As used herein, “neoplasm” or “tumor” (and grammatical variations thereof) means new and abnormal growth of tissue, which may be benign or cancerous. In a related aspect, the neoplasm is indicative of a neoplastic disease or disorder, including but not limited, to various cancers. For example, such cancers can include prostate, pancreatic, biliary, colon, rectal, liver, kidney, lung, testicular, breast, ovarian, brain, and head and neck cancers, melanoma, sarcoma, multiple myeloma, leukemia, lymphoma, or the like.

The amount of the anti-B7-H3 compound administered can vary depending on various factors including, but not limited to, the anti-B7-H3 compound being used, the weight, physical condition, and/or age of the subject, and/or the route of administration. Thus, the absolute weight of anti-B7-H3 compound included in a given unit dosage form can vary widely, and depends upon factors such as the species, age, weight, and physical condition of the subject, and/or the method of administration. Accordingly, it is not practical to set forth generally the amount that constitutes an amount of anti-B7-Compound effective for all possible applications. Those of ordinary skill in the art, however, can readily determine the appropriate amount with due consideration of such factors.

In one or more embodiments, the method can include administering a sufficient amount of the anti-B7-H3 compound to provide a dose of, for example, from about 100 ng/kg to about 50 mg/kg to the subject, although in some embodiments the methods may be performed by administering an anti-B7-H3 compound in a dose outside this range. In some of these embodiments, the method includes administering a sufficient amount of an anti-B7-H3 compound to provide a dose of from about 10 μg/kg to about 5 mg/kg to the subject, for example, a dose of from about 100 μg/kg to about 1 mg/kg.

A single dose may be administered all at once, continuously for a prescribed period of time, or in multiple discrete administrations. When multiple administrations are used, the amount of each administration may be the same or different. For example, a dose of 1 mg per day may be administered as a single administration of 1 mg continuously over 24 hours, as two or more equal administrations (e.g., two 0.5 mg administrations), or as two or more unequal administrations (e.g., a first administration of 0.75 mg followed by a second administration of 0.25 mg). When multiple administrations are used to deliver a single dose, the interval between administrations may be the same or different.

In one or more embodiments, the active ingredient may be administered, for example, from a single dose to multiple doses per week, although in some embodiments the method can involve a course of treatment that includes administering doses of the active ingredient at a frequency outside this range. When a course of treatment involves administering multiple doses within a certain period, the amount of each dose may be the same or different. For example, a course of treatment can include a loading dose (e.g., initial dose), followed by a maintenance dose that is lower than the loading dose. Also, when multiple doses are used within a certain period, the interval between doses may be the same or be different.

In certain embodiments, an anti-B7-H3 compound may be administered from about once per month to about five times per week.

An anti-B7-H3 compound may be administered before, during, or after the subject first exhibits a symptom or clinical sign of the condition. Treatment initiated before the subject first exhibits a symptom or clinical sign associated with the condition may result in decreasing the likelihood that the subject experiences clinical evidence of the condition compared to a subject to which the anti-B7-H3 compound is not administered, decreasing the severity of symptoms and/or clinical signs of the condition, and/or completely resolving the condition. Treatment initiated after the subject first exhibits a symptom or clinical sign associated with the condition may result in decreasing the severity of symptoms and/or clinical signs of the condition compared to a subject to which the composition is not administered, and/or completely resolving the condition.

The anti-B7-H3 compound can be any embodiment of the anti-B7-H3 compound described herein having an anti-B7-H3 affibody that binds to the extracellular domain of B7-H3 displayed on the target cells of a cell population. In some cases, the target cell can include a tumor cell so that the method can involve treating cancer associated with the tumor cells. Thus, in one or more embodiments, the method can include ameliorating at least one symptom or clinical sign of the tumor.

In embodiments in which the target cell includes a tumor cell, the method can further include surgically resecting the tumor and/or reducing the size of the tumor through chemical (e.g., chemotherapeutic) and/or radiation therapy. Exemplary tumors that may be treated include tumors associated with prostate cancer, lung cancer, colon cancer, rectum cancer, urinary bladder cancer, melanoma, kidney cancer, renal cancer, oral cavity cancer, pharynx cancer, pancreas cancer, uterine cancer, thyroid cancer, skin cancer, head and neck cancer, cervical cancer, ovarian cancer, and/or hematopoietic cancer.

In one or more embodiments, the anti-B7-H3 compound is administered prior to, simultaneously with, or following chemotherapy, surgical resection of a tumor, or radiation therapy.

In one or more embodiments, an anti-B7-H3 compound may be administered, for example, from a single dose to multiple doses per week, although in some embodiments the method can be performed by administering an anti-B7-H3 compound at a frequency outside this range. In certain embodiments, an anti-B7-H3 compound may be administered from about once per month to about five times per week.

In one or more embodiments, the method further includes administering one or more additional therapeutic agents. The one or more additional therapeutic agents may be administered before, after, and/or coincident to the administration of an anti-B7-H3 compound. an anti-B7-H3 compound and the additional therapeutic agents may be co-administered. As used herein, the term “co-administered” refers to two or more components of a combination administered so that the therapeutic or prophylactic effects of the combination can be greater than the therapeutic or prophylactic effects of either component administered alone. Two components may be co-administered simultaneously or sequentially. Simultaneously co-administered components may be provided in one or more pharmaceutical compositions. Sequential co-administration of two or more components includes cases in which the components are administered so that each component can be present at the treatment site at the same time. Alternatively, sequential co-administration of two components can include cases in which at least one component has been cleared from a treatment site, but at least one cellular effect of administering the component (e.g., cytokine production, activation of a certain cell population, etc.) persists at the treatment site until one or more additional components are administered to the treatment site. Thus, a co-administered combination can, in certain circumstances, include components that never exist in a chemical mixture with one another. In other embodiments, an anti-B7-H3 compound and the additional therapeutic agent may be administered as part of a mixture or cocktail. In some aspects, the administration of an anti-B7-H3 compound may allow for the effectiveness of a lower dosage of other therapeutic modalities when compared to the administration of the other therapeutic agent or agents alone, thereby decreasing the likelihood, severity, and/or extent of the toxicity observed when a higher dose of the other therapeutic agent or agents is administered. The term “chemotherapeutic agent” as used herein refers to any therapeutic agent used to treat cancer. Examples of chemotherapeutic agents include, but are not limited to, actinomycin, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vinblastine, vincristine, vindesine, vinorelbine, panitumamab, Erbitux™ (cetuximab), matuzumab, IMC-IIF 8, TheraCIM hR3, denosumab, Avastin™ (bevacizumab), Humira™ (adalimumab), Herceptin™ (trastuzumab), Remicade™ (infliximab), rituximab, Synagis™ (palivizumab), Mylotarg™ (gemtuzumab oxogamicin), Raptiva™ (efalizumab), Tysabri™ (natalizumab), Zenapax™ (dacliximab), NeutroSpec™ (Technetium (99mTc) fanolesomab), tocilizumab, ProstaScint™ (Indium-Ill labeled Capromab Pendetide), Bexxar™ (tositumomab), Zevalin™ (ibritumomab tiuxetan (IDEC-Y2B8) conjugated to yttrium 90), Xolair™ (omalizumab), MabThera™ (Rituximab), ReoPro™ (abciximab), MabCampath™ (alemtuzumab), Simulect™ (basiliximab), LeukoScan™ (sulesomab), CEA-Scan™ (arcitumomab), Verluma™ (nofetumomab), Panorex™ (edrecolomab), alemtuzumab, CDP 870, natalizumab, Gilotrif™ (afatinib), Lynparza™ (olaparib), Perjeta™ (pertuzumab), Otdivo™ (nivolumab), Bosulif™ (bosutinib), Cabometyx™ (cabozantinib), Ogivri™ (trastuzumab-dkst), Sutent™ (sunitinib malate), Adcetris™ (brentuximab vedotin), Alecensa™ (alectinib), Calquence™ (acalabrutinib), Yescarta™ (ciloleucel), Verzenio™ (abemaciclib), Keytruda™ (pembrolizumab), Aliqopa™ (copanlisib), Nerlynx™ (neratinib), Imfinzi™ (durvalumab), Darzalex™ (daratumumab), Tecentriq™ (atezolizumab), or Tarceva™ (erlotinib). Examples of immunotherapeutic agents include, but are not limited to, an interleukin (IL-2, IL-7, IL-12, etc.), a cytokine (an interferon, G-CSF, etc.), a chemokine (CCL3, CC126, CXCL7), or an immunomodulatory imide drug (imiquimod, thalidomide, etc., or an analog thereof).

In one or more embodiments, the method can include administering a sufficient amount of an anti-B7-H3 compound as described herein and administering the at least one additional therapeutic agent demonstrates therapeutic synergy. In some aspects of the methods of the present disclosure, a measurement of response to treatment observed after administering both an anti-B7-H3 compound as described herein, and the additional therapeutic agent is improved over the same measurement of response to treatment observed after administering either the anti-B7-H3 compound or the additional therapeutic agent alone.

In yet another aspect, this disclosure describes a capture assay device including any embodiment of one of the anti-B7-H3 compound (including an anti-B7-H3 affibody that does not include an additional functional component) described herein immobilized to a substrate. For example, an anti-B7-H3 compound described herein can be incorporated into cell and/or ligand capture technology such as, for example, an ELISA-based assay. A substrate to immobilize the anti-B7-H3 compound can include, for example, a cell culture plate or dish, a glass slide, or any other support than can be used to perform an assay requiring an immobilized anti-B7-H3 compound.

In another aspect, the present disclosure describes a method for using anti-B7-H3 compounds in molecular imaging applications including, for example, both traditional molecular imaging techniques (e.g., magnetic resonance imaging (MRI), positron emission tomography (PET), single photon emission computed tomography (SPECT), ultrasound, photoacoustic, and fluorescence) and microscopy and/or nanoscopy imaging techniques (e.g., total internal reflection fluorescence (TIRF)-microscopy, stimulated emission depletion (STRED)-nanoscopy, or atomic force microscopy (AFM).

The anti-B7-H3 compounds described herein may have in vitro and in vivo detection, diagnostic, and/or therapeutic utilities. For example, anti-B7-H3 compounds may be included in a detection composition for use in a detection method. The detection composition may include any carrier and/or adjuvant as described herein, or any additional carrier or adjuvant known in the art. The method generally can include allowing an anti-B7-H3 compound that specifically binds to a target of interest with a sample that includes the target of interest, then detecting the formation of an anti-B7-H3 compound: target complex. Thus, the anti-B7-H3 compounds may be designed to include a detectable marker such as, for example, a radioactive isotope, a fluorescent marker, an enzyme, a magnetic marker, or a colorimetric marker.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Reference throughout this specification to “one embodiment,” “one or more embodiments,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be conducted in any feasible order. And, as appropriate, any combination of two or more steps may be conducted simultaneously.

The terms “administration of” and or “administering” should be understood to mean providing a pharmaceutical composition in a therapeutically effective amount to the subject in need of treatment.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

The term “polypeptide” refers to a sequence of amino acid residues without regard to the length of the sequence. Therefore, the term “polypeptide” refers to any amino acid sequence having at least two amino acids and includes full-length proteins, fragments thereof, and/or, as the case may be, polyproteins.

The term “protein” refers to any sequence of two or more amino acid residues without regard to the length of the sequence, as well as any complex of two or more separately translated amino acid sequences. Protein also refers to amino acid sequences chemically modified to include a carbohydrate, a lipid, a nucleotide sequence, or any combination of carbohydrates, lipids, and/or nucleotide sequences. As used herein, “protein,” “peptide,” and “polypeptide” are used interchangeably.

As used herein, the term “nucleic acid” or “oligonucleotide” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugates, and oligonucleotides. A nucleic acid may be single-stranded, double-stranded, linear, or covalently circularly closed molecule. A nucleic acid can be isolated. The term “isolated nucleic acid” means, that the nucleic acid (i) was amplified in vitro, for example via polymerase chain reaction (PCR), (ii) was produced recombinantly by cloning, (iii) was purified, for example, by cleavage and separation by gel electrophoresis, (iv) was synthesized, for example, by chemical synthesis, or (vi) extracted from a sample. A nucleic might be introduced—i.e., transfected-into cells. When RNA is used to transfect cells, the RNA may be modified by stabilizing modifications, capping, or polyadenylation.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Library Design

Affibody library design followed Woldring's gradient site-wise library design, where amino acid diversity at likely hotspot positions was guided by amino acid prevalence in natural antibody interfaces and previously evolved affibodies (Woldring et al., Biochemistry 56, 1656-1671 (2017)).

### Discovery and Evolutionary Strategy

Yeast surface display was used to display affibody libraries, and B7-H3 binders were selected using both magnetic-activated cell sorting (MACS) and fluorescence-activated cell sorting (FACS). Populations were sorted to achieve detectable binding at 50 nM B7-H3 target. DNA was then isolated from yeast and subject to random mutagenesis via error-prone polymerase chain reaction (PCR) of paratope and entire affibody gene. DNA was electroporated back into yeast for display and further sorted until binding was observed to low nanomolar concentrations of B7-H3.

MACS Selections with Soluble Extracellular Domains

Magnetic bead selections were carried out using at least 15-fold oversampling of affibody diversity at all stages. Biotinylated recombinant human B7-H3 extracellular domain (available from Sino Biological, Inc., Beijing, China) for positive isolation and biotinylated Renilla reniformis green fluorescent protein (rrGFP; available from Avidity, LLC, Aurora, CO) for depletion were incubated with DYNABEADS Biotin Binder (Invitrogen, Thermo Fisher Scientific, Inc., Waltham, MA) to coat the beads with protein. Yeast underwent magnetic activated cell sorting, where yeast were incubated with control bare biotin binder beads for two hours at 4° C., followed by another two-hour incubation for depletion with GFP-labeled beads, to remove any non-specific and non-B7-H3 binding interactions. Yeast were then incubated two hours more with beads with immobilized recombinant human B7-H3 target protein. Bound yeast were positively selected. MACS was performed at 4° C., and yeast were washed twice between incubations. A total of three MACS selections were performed with increasing wash stringency, where yeast were grown and induced between each sort. After error-prone PCR, an additional MACS sort was performed on the large mutated naïve library, akin to the procedure described previously.

FACS Selections with Soluble Extracellular Domains

After three MACS sorts of affibody libraries, library size was easily sampleable via FACS. Induced yeast populations were simultaneously labeled with mouse anti-c-Myc antibody (BioLegend, San Diego, CA) and 50 nM biotinylated recombinant human B7-H3 extracellular domain for 30 minutes at 4° C. Cells were washed once with PBSA, labeled with goat anti-mouse Alexa Fluor 647 conjugate (Thermo Fisher Scientific, Inc., Waltham, MA) and streptavidin Alexa Fluor 488 conjugate (Thermo Fisher Scientific, Inc., Waltham, MA) for 20 minutes at 4° C., and washed with PBSA. Cells that were Myc positive and had B7-H3 signal above background were collected.

### Error-Prone PCR of Affibody

Random mutation of affibody was performed by error-prone PCR with mutagenic analogs, 8-oxo-dGTP and dPTP. Zymoprepped plasmid DNA was mutated by error-prone PCR of full affibody genes using primers W5/W3, affibody helices using primers ABY1F-b/ABYIR and ABY2F/ABY2R-b19. PCR products were purified by agarose gel electrophoresis. Affibody helix genes were assembled into one construct using PCR assembly. Final gene inserts were amplified by PCR, concentrated by ethanol precipitation, and resuspended for electroporation. Mutated affibody libraries were homologously recombined with linearized pCT-40-Helix for affibody helix into EBY100 yeast by electroporation transformation. Electroporation yielded roughly 200 million transformants for affibody libraries.

Monovalent MACS Sorting with Soluble Extracellular Target

After a MACS sort was performed on the large mutated naïve library, yeast underwent a more stringent monovalent MACS sort. Yeast were incubated with control bare biotin binder beads, washed, and then depletion by GFP-coated beads, as before. Yeast were then washed and incubated with 100 nM biotinylated recombinant human B7-H3 extracellular domain for one hour. Bare Biotin binder beads were spiked in to bind to biotinylated B7-H3 and were incubated for an additional two hours at 4° C. Beads were washed three times, and yeast remaining bound were collected.

### Preparation of Detergent Solubilized MS1-B7-H3 Cell Lysate

Mile Sven 1 cells stably transfected to express human B7-H3 (MS1-B7-H3) were grown at 37° C. with 5% CO₂ in DMEM with 10% fetal bovine serum (v/v) and 1% penicillin and streptomycin. MS1-B7-H3 cells were grown to 70-90% confluence in 75 cm² tissue culture-treated flasks. Cells were washed with PBS and detached with trypsin-EDTA treatment for five minutes, quenched with serum containing culture media, and pelleted at 500×g for three minutes. Pelleted cells were washed twice and resuspended in PBS with 0.5 mg/mL fresh sulfo-NHS-biotin (Thermo Fisher Scientific, Inc., Waltham, MA) for 30 minutes at room temperature. Cells were washed twice following incubation to remove excess biotin and were lysed in 250 μL lysis buffer for 15 minutes at 4° C. Cell debris was pelleted for 30 minutes at 10,000×g and removed.

FACS Selections with Detergent Solubilized Cell Lysates

Affibody libraries underwent two rounds of flow cytometry selections with detergent solubilized cell lysate. The libraries were washed once with PBSA and incubated with cell lysate for one hour at 4° C. Following incubation, yeast were washed, incubated with chicken anti-Myc-FITC (Immunology Consultants Laboratory Inc., Portland, OR) and streptavidin, Alexa Fluor 647 conjugate (Thermo Fisher Scientific, Inc., Waltham, MA) for 20 minutes at 4° C., and washed again. Yeast that were Myc positive (FITC) with the highest ratio of MS1-B7-H3 lysate binding (AF647): Myc (FITC) were collected using FACS. This sort was repeated with higher stringency by using a lower B7-H3 lysate concentration.

FACS Selection with Protease and Thermostability Assay Yeast induced to display affibody were washed and incubated with proteinase K (1×10⁻⁶ Units/μL; available from New England Biolabs, Inc., Ipswich, MA) for 10 minutes at 55° C., then immediately put on ice. Yeast were then incubated with MS1-B7-H3 cell lysate for one hour at 4° C. Following incubation, yeast were washed, incubated with chicken anti-Myc-FITC and streptavidin-AlexaFluor 647 conjugate for 20 minutes at 4° C., and washed again. Yeast that were Myc positive (FITC) with the highest ratio of MS1-B7-H3 lysate binding (AF647): Myc (FITC) were collected using FACS.

### Sanger Sequencing of Yeast PCR Product

Enriched B7-H3-binding populations were plated on SD-CAA plates and grown for two days. Ten colonies from the affibody library were stochastically chosen and incubated at 100° C. for five minutes in 50 μL of distilled water. Two microliters of yeast sample were taken and underwent PCR with GeneAmp5/3 primers and DNA clean up. Amplified affibody genes and GeneAmp5 primer were sent to Eurofins Genomics LLC for Sanger sequencing.

### Cloning, Protein Expression, and Purification

Affibody encoding regions in DNA recovered from the final B7-H3 flow cytometry sort were amplified by PCR, digested with NheI-HF and BamHI-HF restriction enzymes (New England Biolabs, Inc., Ipswich, MA) and ligated with T4 DNA ligase into pET-24b vector containing a C-terminal hexa-histidine tag. Plasmids were transformed into T7 Express Competent E. coli and plated on lysogeny broth (LB) plates containing 50 mg/L kanamycin. Transformants were Sanger sequenced for full-length gene and proper transformants were grown in 5 mL liquid LB with kanamycin (50 mg/L) at 37° C. at 250 rpm for 12-16 hours. Saturated cultures were added to 100 mL LB, grown, and induced. Cells were pelleted and resuspended in lysis buffer (50 mM sodium phosphate (pH 8.0), 0.5 M sodium chloride, 5% glycerol, 5 mM 3-[(3-cholamidopropyl)dimethylammonio]-1-propanesulfonate, and 25 mM imidazole), frozen and thawed five times to lyse cells, centrifuged for 10 minutes at 4° C., and 0.25 mm filtered. The resulting cell lysates were run through 0.25 mL Cobalt HisPur resin volume spin columns, washed with 30 mM imidazole, and eluted with 300 mM imidazole. Affibody purity and concentration were analyzed via protein gel electrophoresis.

### Affinity Titrations

Detached MS1-B7-H3 cells were washed and individually labeled with varying concentrations of purified affibody and affibody fusions for at least 30 minutes at 4° C. Cells were pelleted at 500×g for three minutes and washed with cold PBSA prior to labeling with anti-His6 FITC conjugate (available from Abcam, Cambridge, United Kingdom) for 20 minutes at 4° C. Fluorescence was analyzed using ACCURI C6 Plus. The dissociation constant was calculated by nonlinear least-squares regression using a 1:1 binding model in PRISM software (GraphPad Software, San Diego, CA).

### Analyzing Modularity of Affibodies

Affibody gene encoding regions were amplified by PCR and assembled via NEBuilder HiFi DNA Assembly (New England Biolabs, Inc., Ipswich, MA) into pET-24b vector containing a GSGGGSGGGKGGGGT (SEQ ID NO:35) linker and amino acids 60-206 of SortaseA (EC 3.4.22.70) gene with a C-terminal hexa-histidine tag. Plasmids were transformed into BL21-CodonPlus (DE3)-RIL competent E. coli and plated on LB plates containing 50 mg/L kanamycin. Transformants were Sanger sequenced for full-length gene and proper transformants were grown in 50 mg/L kanamycin and chloramphenicol for bacteria growth and protein purification. Purified proteins were affinity titrated to assess binding affinity of select affibodies in presence of conjugated protein enzyme.

### Example 2

Example 2 describes affinity maturation, specificity maturation, and stability maturation of the 1.5 affibody population of Example 1.

### Error-Prone PCR

DNA was isolated from the 1.5 yeast population and subjected to random mutagenesis via error-prone polymerase chain reaction (PCR) of variable regions of the affibodies. DNA was electroporated back into yeast for display and further sorted using the following sort scheme: (1) an avidity sort (lysate MACS with 1 pmol B7-H3+ lysate); (2) an affinity sort (strict K_{d} FACS sort); (3) specificity sort (multitarget MACS depletion sort); and (4) stability sort (thermolysin at 55° C. FACS sort). After each sort, the populations were deep sequenced, filtered via USEARCH (ultra-fast sequencing analysis; Robert C. Edgar, Search and clustering orders of magnitude faster than BLAST, Bioinformatics, Volume 26, Issue 19, 1 Oct. 2010, Pages 2460-2461, doi.org/10.1093/bioinformatics/btq461), and filtered for full length affibody variants.

The frequencies at each sort (e.g., avidity, affinity, specificity, and stability) were determined for each sequence, as well as their subsequent enrichment. If read count was 0, a read count of 1 was assigned. The z-score for affinity, specificity, stability, and final frequency was determined and a final conditional, weighted averaged z-score was calculated. Top performers were selected based on the highest score.

The conditional, weighted average z-score was calculated by first determining the frequency of each variant in each sort. The enrichment values for affinity, specificity, and stability sorts for each variant were then calculated by dividing the frequency of single variant at specific sort by its frequency in the previous sort. Its z-score was calculated by (sort enrichment score-average sort enrichment score of all variants) divided by (standard deviation of sort enrichment score of all variants). The z-scores were averaged based on the following weights: affinity=1; frequency=3; specificity=2; and stability=1. The final frequency and specificity z-scores were most important, as these were given the greatest weight. Additionally, the following conditions were also included: affinity enrichment >0.5 and final frequency >0. Affinity enrichment values greater than 1 indicate enrichment. A value of 0.5 was chosen to account for variants that were initially present at higher frequencies and may not have had opportunity to enrich. The final frequency condition was included because variants must be present in final sequenced population.

FIG. 6 shows the z-scores of affinity, specificity, stability, and final frequency as well as the conditional weighted average z-score for the top 13 variants. Values greater than 1 indicate enrichment and values less than 1 indicate depletion. The color bars in FIG. 6 for affinity range M−M+2SD enrichment, while specificity and stability are displayed 1−M+2SD, as the mean (M) was slightly less than 1. On the bar chart of FIG. 6, the z-score is displayed 1-10 (i.e., if the z-score is 10 or above, the bar spans the entire length of the chart).

### B7-H3 Cell Lysate MACS Sort (Avidity Sort):

An avidity sort was conducted. This sort is considered an avidity sort because the B7-H3 target is expressed multivalently on avidin beads, not just monovalently in solution. The yeast are also expressing thousands of affibodies per cell, so avid binding can occur across yeast and B7-H3-coated beads.

Magnetic bead selection was performed with 20-fold oversampling to select for B7-H3 binders and easily weed out non-binders/truncated affibodies resulting from error prone PCR. Yeast were incubated with bare streptavidin-coated beads for two hours at 4° C., followed by another two-hour incubation for depletion with GFP-coated beads. Yeast were incubated two hours more with 1 pmol of detergent solubilized biotinylated MS1-B7-H3 cell lysate. Usually, 33 pmol of antigen are used to coat the streptavidin beads; however, 1 pmol was chosen to be more stringent (e.g., decrease avidity). Bound yeast were selected and grown. Biotinylation of the MS1-B7-H3 cell lysate is non-specific and reacts to all proteins (non-specific N-hydoxysuccinimide (NHS) chemistry), not just B7-H3. B7-H3 is expressed at ˜1 million per cell and the affibody libraries have already been primed to have binding to B7-H3.

### Strict K_{d} FACS Sort (Affinity Sort)

After one round of the B7-H3 cell lysate MACS sort, library size was easily sampleable via FACS. Induced yeast populations were labeled with ˜0.2 nM biotinylated-B7-H3 cell lysate for two hours, washed, and labeled with streptavidin-AF647 and chicken anti-Myc-FITC. The top 0.4% of cells (highest affinity) was collected for the next sort (the specificity sort).

### Multitarget MACS Depletion (Specificity Sort)

To select for more specific binders, the population isolated after the affinity sort underwent a multitarget depletion. The following proteins were biotinylated, incubated with streptavidin-coated beads, and used for negative depletion: lysozyme, bovine serum albumin, human interleukin receptor 2 gamma, tobacco etch virus (TEV) protease, human plasminogen activator urokinase receptor, human carbonic anhydrase II, and rabbit IgG-FITC. These proteins were used because they were readily available and/or already conjugated to biotin. Yeast were incubated with the aforementioned proteins coated on beads for two hours at 4° C. Yeast then underwent the same negative depletion step a second time. This was to increase confidence that non-specific binders were depleted. Non-binding yeast were selected and grown for the next sort (the stability sort).

### Thermolysin Stability FACS Sort (Stability Sort)

To select for stable binders, the affibody population after the specificity sort underwent a protease incubation at increased temperature. Yeast were induced to display the affibody population and were incubated with 0.75 mg/mL thermolysin for 10 minutes at 55° C., then immediately put on ice. Yeast were washed and incubated with mouse anti-MYC and rabbit anti-HA-biotin for 20 minutes. Yeast were again washed and labeled with goat anti-mouse-AF647 and streptavidin-FITC for another 20 minutes. Yeast that were HA and Myc positive were sorted in a three-tiered gating approach, where the top 2%, 14%, and 19% displaying yeast were collected using FACS.

### Deep Sequencing Via ISEQ (Illumina, San Diego, CA)

Sorted affibody populations were deep sequenced, filtered via USearch, and further filtered for full length affibody. In the stability sort, three tiered gates were collected and sequenced. Due to lack of sequencing depth of full-length affibodies, the read counts from each of the stability gates were summed for a total stability read count. Clones that recapitulated a previously engineered B7-H3 affibody were removed for sequence analysis.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

### Sequence Listing Free Text

ABY
SEQ ID NO: 1
AEAKYAKEQQ NAFYEILHLP NLTEEQRNAF IQKLKDDPSQ SSELLSEAKKL NDSQAPK
General Consensus Sequence
SEQ ID NO: 2
AEAKYXKEXX XAXXXIXXLP NLXXXQXXAF XXXLXXDPSQ SSELXXEAXK LNDSQAPK
X₆: Y, N, S, T, D, or A;
X₉: L, R, K, W, D, I, N, or Q;
X₁₀: H, A, S, F, I, D, E, Q, T, N, or L;
X₁₁: T, Y, D, A, L, P, I, or N;
X₁₃: I, Y, L, V, T, or F;
X₁₄: Q, Y, L, V, G, F, E, A, M, or S;
X₁₅: V, S, Q, or E;
X₁₇: H, A, V, G, R, I, F, or L;
X₁₈: S, Y, L, G, T, N, M, D, R, W, E, or H;
X₂₃: T or A;
X₂₄: L, K, A, G, R, H, W, V, or E;
X₂₅: D, P, T, N, V, S, H, L, R, A, W, or E;
X₂₇: R, K, T, I, or A;
X₂₈: A, Y, R, N, D, Q, I, H, L, S, or F;
X₃₁: L, K, I, or T;
X₃₂: L, G, Y, R, A, F, V, S, or Q;
X₃₃: A or K;
X₃₅: F, S, V, H, Y, D, E, or K;
X₃₆: N or D;
X₄₄: L or F;
X₄₆: S or P;
X₄₉: R or K.
Second consensus sequence for SEQ ID NO: 4 through SEQ ID NO: 18
SEQ ID NO: 3
AEAKYYKELH NAIGVIRSLP NLTRDQRAAF LLALENDPSQ SSELLSEAKKL NDSQAPK
ABY 0.4.1
SEQ ID NO: 4
AEAKYYKELH NAIQVIHYLP NLTLDQKYAF KLALFNDPSQ SSELLSEAKK LNDSQAPK
ABY 0.4.2
SEQ ID NO: 5
AEAKYNKELA TAYYVIALLP NLTEPQRAAF LGALSDDPSQ SSELLSEAKK LNDSQAPK
ABY 0.4.3
SEQ ID NO: 6
AEAKYYKELH TAILSIHGLP NLTKTQKRAF IYALFNDPSQ SSELLSEAKK LNDSQAPK
ABY 0.4.4
SEQ ID NO: 7
AEAKYNKERS YAIYQIVLLP NLTANQRNAF LRALVDDPSQ SSELLSEAK KLNDSQAPK
ABY 0.4.5
SEQ ID NO: 8
AEAKYSKEKF DAILSILSLP NLTAVQRDAF LGALHDDPSQ SSELLSEAKK LNDSQAPK
ABY 0.4.6
SEQ ID NO: 9
AEAKYYKELH YAIVSIRGLP NLTASQRIAF LFALENDPSQ SSELLSEAKK LNDSQAPK
ABY 0.4.7
SEQ ID NO: 10
AEAKYTKEWI AAYLQIGYLP NLTGVQRQAF TAALYNDPSQ SSELLSEAKK LNDSQAPK
ABY 1.4.1
SEQ ID NO: 11
AEAKYDKEKS NAILSIVTLP NLTRVQRHAF LVALHNDPSQ SSELLSEAKK LNDSQAPK
ABY 1.4.2
SEQ ID NO: 12
AEAKYYKELH DAIVQIRNLP NLTRPQRAAF KLALENDPSQ SSELLSEAKK LNDSQAPK
ABY 1.4.3
SEQ ID NO: 13
AEAKYYKELH NAIGVIRSLP NLTRHQRAAF LLALENDPSQ SSELLSEAKK LNDSQAPK
ABY 1.4.4
SEQ ID NO: 14
AEAKYDKERL NAIFQIVLLP NLTRDQKLAF TAALYDDPSQ SSELLSEAKK LNDSQAPK
ABY 1.4.5
SEQ ID NO: 15
AEAKYYKELH NAIQVIHYLP NLTRDQKYAF KLALENDPSQ SSELLSEAKK LNDSQAPK
ABY 1.4.6
SEQ ID NO: 16
AEAKYYKELH NAIGVIRSLP NLTRDQRYAF KLALFNDPSQ SSELLSEAKK LNDSQAPK
ABY 1.4.7
SEQ ID NO: 17
AEAKYYKELH NAIGVIRSLP NLTHHQRSAF LLALENDPSQ SSELLSEARK LNDSQAPK
ABY 1.4.8
SEQ ID NO: 18
AEAKYYKELH NAIGVIRSLP NLTHHQKSAF LLALENDPSQ SSELLPEAKK LNDSQAPK
ABY 1.5.1
SEQ ID NO: 19
AEAKYYKELH NAIGVIRSLP NLTRHQRAAF LLALENDPSQ SSELLSEAKKL NDSQAPK
ABY 1.5.2
SEQ ID NO: 20
AEAKYYKELH DAIQVIHYLP NLTLDQKYAF KLALENDPSQ SSELLSEAKKL NDSQAPK
Otu13
SEQ ID NO: 22
AEAKYAKELD NAYYEIVTLP NLTWLQTFAF IFALDNDPSQ SSELLSEAKK LNDSQAPK
Otu8
SEQ ID NO: 23
AEAKYAKELH NAIGVIRSLP NLTRHQRAAF LLALENDPSQ SSELLSEAKK LNDSQAPK
Otu12
SEQ ID NO: 24
AEAKYYKELH DAIQVIHYLP NLTRRQRAAF KLALENDPSQ SSELLSEAKK LNDSQAPK
Otu15
SEQ ID NO: 25
AEAKYAKEKS NAVLSIVSLP NLTRAQRHAF LVALHNDPSQ SSELLSEAKK LNDSQAPK
Otu11
SEQ ID NO: 26
AEAKYNKEDS NAYEEIIMLP NLTVWQILAF IVALDNDPSQ SSELLSEAKK LNDSQAPK
Otu89
SEQ ID NO: 27
AEAKYDKELE LAYLEIFDLP NLTWLQTFAF ISALENDPSQ SSELLSEAKK LNDSQAPK
Otu227
SEQ ID NO: 28
AEAKYAKELEP ALAEILRLP NLTWLQTIAF ILALEDDPSQ SSELLSEAKK LNDSQAPK
Otu1607
SEQ ID NO: 29
AEAKYYKEKS NAVLSIVSLP NLARAQRAA FLVALHDDPSQ SSEFLSEAKK LNDSQAPK
Otu225
SEQ ID NO: 30
AEAKYAKEDQ NALSEILWLP NLTGWQTFA FILALENDPSQ SSELLSEAKK LNDSQAPK
Otu394
SEQ ID NO: 31
AEAKYYKELH NAIGVIRSLP NLTRHQRTAF LLALENDPSQ SSELLSEAKK LNDSQAPK
Otu249
SEQ ID NO: 32
AEAKYAKEIE IAYVEILLLP NLTWLQTYAF IFALEDDPSQ SSELLSEAKK LNDSQAPK
Otu252
SEQ ID NO: 33
AEAKYAKENT IALMEIIELP NLTLLQTLAF IVALDDDPSQ SSELLSEAKK LNDSQAPK
Otu265
SEQ ID NO: 34
AEAKYDKELN IATMEILTLP NLTLWQAIAF IVALDDDPSQ SSELLSEAKK LNDSQAPK
Linker
SEQ ID NO: 35
GSGGGSGGGK GGGGT
linker
SEQ ID NO: 36
GGGGSGGGGS GGGGS
linker
SEQ ID NO: 37
GSTSGSGKPG SGEGSTKG
linker
SEQ ID NO: 38
PSGQAGAAAS ESLFVSNHAY
linker
SEQ ID NO: 39
EASGGPE
linker
SEQ ID NO: 40
AEAAKEAAKE AAKEAAKALE AEAAKEAAKE AAKEAAKA
linker
SEQ ID NO: 41
AEAAKEAAKA
linker
SEQ ID NO: 42
SGGGGSGGGGS GGGGSGGGGSG
linker
SEQ ID NO: 43
GGGGSGGGGS
linker
SEQ ID NO: 44
GSTSGSGKPG SGEGSTKG
linker
SEQ ID NO: 45
EPKSSDKTHT SPPSP
linker
SEQ ID NO: 46
RATPSHNSHQ VPSAGGPTAN SGTSG
linker
SEQ ID NO: 47
SSGGGGSGGG GGGSSRSSL

1. An anti-B7-H3 compound comprising an anti-B7-H3 affibody, the anti-B7-H3 affibody comprising:
SEQ ID NO:2;
an amino acid sequence having at least 90% sequence similarity to SEQ ID NO:2; or
an amino acid sequence having at least 90% sequence identity to SEQ ID NO:2.

2. The anti-B7-H3 compound of claim 1, wherein the anti-B7-H3 affibody comprises:
any one of SEQ ID NO:3 through SEQ ID NO:20 or SEQ ID NO:22 through SEQ ID NO: 34;
an amino acid sequence having at least 90% sequence similarity to any one of SEQ ID NO: 3 through SEQ ID NO:20 or SEQ ID NO:22 through SEQ ID NO:34; or
an amino acid sequence having at least 90% sequence identity to any one of SEQ ID NO: 3 through SEQ ID NO:20 or SEQ ID NO:22 through SEQ ID NO:34.

3. The anti-B7-H3 compound of claim 1, further comprising a first functional component operably coupled to the anti-B7-H3 affibody.

4. The anti-B7-H3 compound of claim 3, wherein the first functional component comprises a targeting component, an imaging component, an enzyme, or a small molecule drug.

5. The anti-B7-H3 compound of claim 4, wherein the targeting component binds to the extracellular domain of a protein displayed on a natural killer cell or a T cell.

6. The anti-B7-H3 compound of claim 4, wherein the imaging component comprises a colorimetric label, a fluorescent label, a radioactive label, a magnetic label, or an enzymatic label.

7. The anti-B7-H3 compound of claim 4, wherein the enzyme comprises sortase A.

8. The anti-B7-H3 compound of claim 4, wherein the small molecule drug includes one or more radioisotopes.

9. The anti-B7-H3 compound of claim 4, further comprising a second functional component operably coupled to anti-B7-H3 affibody.

10. The anti-B7-H3 compound of claim 9, wherein the second functional component comprises a targeting component, an imaging component, an enzyme, or a small molecule drug.

11. The anti-B7-H3 compound of claim 9, wherein the first functional component is directly linked to the anti-B7-H3 affibody and the second functional component is directly linked to the anti-B7-H3 affibody.

12. The anti-B7-H3 compound of claim 9, wherein the first functional component is directly linked to the anti-B7-H3 affibody and the second functional component is directly linked to the first functional component.

13. A composition comprising:
the anti-B7-H3 compound of claim 1; and
a pharmaceutically acceptable carrier.

14. A method comprising administering the composition of claim 13 to a subject.

15. The method of claim 14, wherein the subject has a tumor or is at risk of having a tumor.

16. The method of claim 14, wherein the subject has cancer or is at risk of having cancer.

17. The method of claim 14, wherein the composition is administered prior to, simultaneously with, or following chemotherapy, surgical resection of a tumor, or radiation therapy.

18. The method of claim 14, further comprising detecting the imaging component to detect anti-B7-H3 compound bound to B7-H3 expressed by a cell.

19. The method of claim 18, wherein the cell is a cancer cell that expressed B7-H3.

20. The method of claim 18, wherein the imaging component can be detected by positron emission tomography.
